SEQUENCE LISTING (23 sequences)

SEQ ID NO: 1 - protein, 9 aa, synthetic construct
  REGION 1..9: note = MAGEA3 (108-116)
ALSRKVAEL

SEQ ID NO: 2 - protein, 9 aa, synthetic construct
  REGION 1..9: note = MAGEA3 (271-279)
FLWGPRALV

SEQ ID NO: 3 - protein, 10 aa, synthetic construct
  REGION 1..10: note = MAGEA4 (230-239)
GCYDGREHTV

SEQ ID NO: 4 - protein, 9 aa, synthetic construct
  REGION 1..9: note = hLMP2 (aa426-434)
CLGGLLTMV

SEQ ID NO: 5 - protein, 9 aa, synthetic construct
  REGION 1..9: note = DNTT (250-258)
KLFTSVFGV

SEQ ID NO: 6 - protein, 9 aa, synthetic construct
  REGION 1..9: note = hPOLM (aa238-246)
KLFTQIFGV

SEQ ID NO: 7 - protein, 9 aa, synthetic construct
  REGION 1..9: note = hPYGL (aa622-630)
KLITSVADV

SEQ ID NO: 8 - protein, 12 aa, synthetic construct
  REGION 1..12: note = Gliadin
QLQPFPQPELPY

SEQ ID NO: 9 - protein, 12 aa, synthetic construct
  REGION 1..12: note = HSV
EEVDMTPADALD

SEQ ID NO: 10 - protein, 12 aa, synthetic construct
  REGION 1..12: note = Gliadin peptide
PQPELPYPQPQL

SEQ ID NO: 11 - protein, 12 aa, synthetic construct
  REGION 1..12: note = Gliadin peptide
FPQPEQPFPWQP

SEQ ID NO: 12 - protein, 5 aa, synthetic construct
  REGION 1..5: note = peptide
GGGGS

SEQ ID NO: 13 - protein, 13 aa, synthetic construct
  REGION 1..13: note = PADRE peptide
AKFVAAWTLKAAA

SEQ ID NO: 14 - protein, 15 aa, synthetic construct
  REGION 1..15: note = LCMV tag
SERPQASGVYMGNLT

SEQ ID NO: 15 - protein, 15 aa, synthetic construct
  REGION 1..15: note = BirA tag
GLNDIFEAQKIEWHE

SEQ ID NO: 16 - protein, 26 aa, synthetic construct
  REGION 1..26: note = mmH tag
EQKLISEEDLEQKLISEEDLHHHHHH

SEQ ID NO: 17 - protein, 28 aa, synthetic construct
  REGION 1..28: note = mmH tag
EQKLISEEDLGGEQKLISEEDLHHHHHH

SEQ ID NO: 18 - protein, 11 aa, synthetic construct
  REGION 1..11: note = Hepatitis B virus polymerase peptide POL606-616
GSLPQEHIVQK

SEQ ID NO: 19 - protein, 9 aa, synthetic construct
  REGION 1..9: note = HIV GAG peptide
AISPRTLNA

SEQ ID NO: 20 - protein, 9 aa, synthetic construct
  REGION 1..9: note = Epstein-Barr virus peptide BZLF1 (40-48)
SQAPLPCVL

SEQ ID NO: 21 - protein, 9 aa, synthetic construct
  REGION 1..9: note = hMAGE-A4 aa 286-294
KVLEHVVRV

SEQ ID NO: 22 - protein, 40 aa, synthetic construct
  REGION 1..40: note = Jun dimerization motif (40 aa)
RIARLEEKVKTLKAQNSELASTANMLREQVAQLKQKVMNH

SEQ ID NO: 23 - protein, 39 aa, synthetic construct
  REGION 1..39: note = Fos dimerization motif (39aa)
LTDTLQAETDQLEDEKSALQTEIANLLKEKEKLEFILAA

(19) United States
(12) Patent Application Publication
(10) Pub. No.: US 2025/0101125 A1
(43) Pub. Date: Mar. 27, 2025

(54) METHODS FOR OBTAINING ANTIBODY MOLECULES BINDING TO A PEPTIDE-MHC INTERFACE

(71) Applicant: Regeneron Pharmaceuticals, Inc., Tarrytown, NY (US)

(72) Inventors: Wen-Yi LEE, New Hyde Park, NY (US); David SUH, Midland Park, NJ (US); Gang CHEN, Yorktown Heights, NY (US)

(73) Assignee: Regeneron Pharmaceuticals, Inc., Tarrytown, NY (US)

(21) Appl. No.: 18/757,640

(22) Filed: Jun. 28, 2024

Related U.S. Application Data
(60) Provisional application No. 63/584,509, filed on Sep. 22, 2023.

(51) Int. Cl. C07K 16/28; C07K 16/08; C07K 16/10; C07K 16/30; C07K 16/40

(52) U.S. Cl. CPC C07K 16/2896; C07K 16/082; C07K 16/084; C07K 16/085; C07K 16/1045; C07K 16/30; C07K 16/3069; C07K 16/40

(57) ABSTRACT

Disclosed herein are methods for isolating antibody-producing cells that express antibody molecules specific for an interface between a target peptide and an MHC molecule. The methods disclosed herein utilize a blocking reagent when sorting cells such as splenocytes which permits enrichment of cells expressing rare antibody molecules that specifically recognize an MHC-peptide interface.

### CROSS REFERENCE TO RELATED APPLICATION

This application claims the benefits of priority from U.S. Provisional Application 63/584,509, filed Sep. 22, 2023, the entire contents of which are incorporated herein by reference.

### INCORPORATION BY REFERENCE TO SEQUENCE LISTING

The sequence listing in the XML file, named as 42347_11577US01_SequenceListing of 29 KB, created on Jun. 25, 2024, and submitted to the United States Patent and Trademark Office via Patent Center, is incorporated herein by reference.

### BACKGROUND

Numerous peptides derived from most, if not all, cellular proteins, including tumor-specific antigens such as melanoma-associated antigen 3 (MAGEA3) and MAGE-A4, are generated by the proteasome and presented on the cell surface by human leukocyte antigen (HLA) molecules corresponding to class I major histocompatibility complexes (MHCs). MAGE3 and MAGE4 peptides presented by the MHC class I allele HLA-A2 are markers for many cancer cells making it a clinically important immunotherapeutic oncology target. In contrast to MHC class I molecules, the antigens presented by MHC class II are derived from extracellular proteins. Isolation of antibodies directed to a specific MHC-peptide interface will serve as a powerful tool for the development of therapeutic antibody molecules to clinically important targets.

### SUMMARY

The use of flow cytometry enables the isolation of antigen-specific B cells, expressing antibodies with specific predefined properties, among hundreds of millions of splenocytes from immunized mice. It has been discovered herein that inclusion of a blocking reagent when sorting cells such as splenocytes permits enrichment of cells expressing rare antibody molecules that specifically recognize an MHC-peptide interface.

One aspect of this disclosure provides a method for isolating antibody-producing cells that express antibody molecules specific for a peptide-MHC interface, the method comprising:
(a) contacting a population of antibody-producing cells encompassing cells that express on the cell surface antibody molecules specific for an interface between a target peptide and an MHC molecule with a blocking reagent, said blocking reagent comprises a peptide-MHC trimer,
(b) contacting the population of cells in the presence of the blocking reagent with a sorting reagent which comprises a labeled peptide-MHC trimer,
(c) wash the population of cells to remove unbound sorting reagent; and
(d) collecting cells that remain bound to the sorting reagent to obtain cells expressing antibody molecules specific for an interface between the target peptide and the MHC molecule,
wherein the peptide-MHC trimer in the blocking reagent comprises a control peptide and two MHC polypeptide chains comprising (i) the α chain or a portion thereof of the MHC molecule and β2 microglobulin or a portion thereof when the MHC molecule is an MHC class I molecule, or (ii) the α chain or a portion thereof and the β chain or a portion thereof of the MHC molecule when the MHC molecule is an MHC class II molecule, wherein the control peptide differs from the target peptide by at least one amino acid, and is presented in a peptide-binding groove formed by the two MHC polypeptide chains;
wherein the peptide-MHC trimer in the sorting reagent comprises the target peptide and two MHC polypeptide chains comprising (i) the α chain or a portion thereof of the MHC molecule and β2 microglobulin or a portion thereof when the MHC molecule is an MHC class I molecule, or (ii) the α chain or a portion thereof and the β chain or a portion thereof of the MHC molecule when the MHC molecule is an MHC class II molecule, wherein the target peptide is presented in a peptide-binding groove formed by the two MHC polypeptide chains; and
wherein the peptide-MHC trimer in the blocking reagent is unlabeled or labeled differently from the labeled peptide-MHC trimer in the sorting reagent.

In some embodiments, the MHC molecule is an MHC class I molecule. In some embodiments, the MHC class I molecule is a human MHC class I molecule. In some embodiments, the human MHC class I molecule is selected from the group consisting of HLA-A, HLA-B, HLA-C and HLA-E. In some embodiments, the MHC molecule is an MHC class II molecule. In some embodiments, the MHC class molecule is a human MHC class II molecule. In some embodiments, the human MHC class II molecule is selected from the group consisting of HLA-DQ, HLA-DR, and HLA-DP.

In embodiments where the MHC molecule is an MHC class I molecule, in the blocking reagent, the α chain or the portion thereof may comprise the extracellular sequence of an MHC class I α chain, and the β2 microglobulin or the portion thereof may comprise the mature form of the β2 microglobulin; and in the sorting reagent, the α chain or the portion thereof may comprise the extracellular sequence of the α chain, and the β2 microglobulin or the portion thereof may comprise the mature form of the β2 microglobulin.

In embodiments where the MHC molecule is an MHC class II molecule, in the blocking reagent, the α chain or the portion thereof may comprise the extracellular sequence of the α chain, and the β chain or the portion thereof may comprise the extracellular sequence of the β chain; and in the sorting reagent, the α chain or the portion thereof may comprise the extracellular sequence of the α chain, and the β chain or the portion thereof may comprise the extracellular sequence of the β chain.

In some embodiments, the two polypeptide chains of the MHC molecule in the peptide-MHC trimer in the blocking reagent are identical in sequence to the two polypeptide chains of the MHC molecule in the peptide-MHC trimer in the sorting reagent. In some embodiments, the two polypeptide chains of the MHC molecule in the peptide-MHC trimer in the blocking reagent are not identical in sequence to the two polypeptide chains of the MHC molecule in the peptide-MHC trimer in the sorting reagent; for example, with a difference being that at least one of the two polypeptide chains of the MHC molecule in the peptide-MHC trimer in the blocking reagent comprises a cysteine residue permitting formation of a disulfide bond in substitution of a non-cysteine amino acid residue in a corresponding polypeptide chain of the MHC molecule in the peptide-MHC trimer in the sorting reagent, or alternatively, at least one of the two polypeptide chains of the MHC molecule in the peptide-MHC trimer in the sorting reagent comprises a cysteine residue permitting formation of a disulfide bond in substitution of a non-cysteine amino acid residue in a corresponding polypeptide chain of the MHC molecule in the peptide-MHC trimer in the blocking reagent.

In some embodiments, the control peptide in the blocking reagent is covalently linked to one of the two MHC polypeptide chains, and/or the target peptide in the sorting reagent is covalently linked to one of the two MHC polypeptide chains. In embodiments where the MHC molecule is an MHC class I molecule, the control peptide in the blocking reagent may be covalently attached to the α chain or the portion thereof or the β2 microglobulin or the portion thereof via a peptide linker. In some embodiments, the control peptide, the α chain or the portion thereof, and the β2 microglobulin or the portion thereof, are linked in a single chain. In embodiments where the MHC molecule is an MHC class I molecule, the target peptide in the sorting reagent may be covalently attached to the α chain or the portion thereof or the β2 microglobulin or the portion thereof via a peptide linker. In some embodiments, the target peptide, the α chain or the portion thereof, and the β2 microglobulin or the portion thereof, are linked in a single chain.

In embodiments where the MHC molecule is an MHC class II molecule, the control peptide in the blocking reagent may be covalently attached to the α chain or the portion thereof or the β chain or the portion thereof via a peptide linker. In some embodiments, the α chain or the portion thereof and the β chain or the portion thereof are linked by a Jun-Fos zipper comprising a Jun leucine zipper dimerization motif and a Fos leucine zipper dimerization motif. In embodiments where the MHC molecule is an MHC class II molecule, the target peptide in the sorting reagent may be covalently attached to the α chain or the portion thereof or the β chain or the portion thereof via a peptide linker. In some embodiments, the α chain or the portion thereof, and the β chain or the portion thereof, are linked by a Jun-Fos zipper comprising a Jun leucine zipper dimerization motif and a Fos leucine zipper dimerization motif.

In some embodiments, the target peptide comprises a tumor associated antigen, a bacterial antigen, or a viral antigen. In some embodiments, the tumor associated antigen is derived from a mutated oncogene such as a KRAS neoantigens, a viral oncogene such as HPV E7, a lineage differentiation antigen (such as melanocyte antigens like gp100), an oncofetal antigen such as WT-1, and a cancer-testis antigen (CTA). In some embodiments, the tumor associated antigen is derived from a protein of the MAGE family such as MAGE-A3 or a MAGE-A4. In some embodiments, the tumor associated antigen is a peptide fragment of DNA nucleotidylexotransferase (DNTT). In some embodiments, a viral antigen comprises a peptide of a protein of Hepatitis B virus, HIV, or Epstein-Barr virus.

In some embodiments, the control peptide differs from the target peptide by at least 2 amino acids. In some embodiments, the control peptide differs from the target peptide by at least 3 amino acids, e.g., 3, 4, 5, 6, 7, 8 or more amino acids. In some embodiments, the control peptide differs from the target peptide in at least 10% of the amino acids. In some embodiments, the control peptide is an off-target peptide.

In some embodiments, the blocking reagent comprises two or more peptide-MHC trimers, wherein the control peptides in the two or more peptide-MHC trimers differ from one another.

In some embodiments, the peptide-MHC trimer in the blocking reagent is unlabeled. In some embodiments, the unlabeled peptide-MHC trimer in the blocking reagent in step (a) has a molar concentration at least 10 fold relative to the molar concentration of the labeled peptide-MHC trimer in the sorting reagent. In some embodiments, the unlabeled peptide-MHC trimer in the blocking reagent in step (a) has a molar concentration at least 50 fold relative to the molar concentration of the labeled peptide-MHC trimer in the sorting reagent. In some embodiments, the unlabeled peptide-MHC trimer in the blocking reagent in step (a) has a molar concentration at least 100 fold relative to the molar concentration of the labeled peptide-MHC trimer in the sorting reagent.

In some embodiments, the peptide-MHC trimer(s) in the blocking reagent is(are) labeled differently from the peptide-MHC trimer in the sorting reagent. In some embodiments, the peptide-MHC trimer in the sorting reagent is labeled with a first fluorescent compound, and wherein the peptide-MHC trimer(s) in the blocking reagent is(are) labeled with a second fluorescent compound that differentiates from the first fluorescent compound. In some embodiments, the peptide-MHC trimer in the blocking reagent is at about the same (or comparable) or higher molar concentration as the peptide-MHC trimer in the sorting reagent.

In some embodiments, the blocking reagent and the sorting reagent are brought into contact with the population of cells at the same time. In some embodiments, the blocking reagent is brought into contact with the population of cells first, followed by the sorting reagent.

In some embodiments, the peptide-MHC trimer in the sorting reagent is labeled with indirectly with a fluorescent compound. For example, the peptide-MHC trimer in the sorting reagent may be conjugated with biotin which binds streptavidin labeled with the fluorescent compound.

In some embodiments, the peptide-MHC trimer in the blocking reagent and/or the sorting reagent is provided in a multimeric form, for example, a dimer, trimer, tetramer, pentamer, hexamer of the peptide-MHC trimer, or a mixture thereof. In some embodiments, the peptide-MHC trimer in the blocking reagent and/or the sorting reagent is provided in both monomeric and multimeric forms. In embodiments where a multimeric form of a peptide-MHC trimer is employed, the multimeric form can be formed by utilizing a multivalent molecule to which the peptide-MHC trimer is bound or linked. In some embodiments, the multivalent molecule is a streptavidin multimer (e.g., tetramer), which can be bound by biotin to which the peptide-MHC trimer is conjugated; and in some such embodiments the streptavidin can be labeled with a fluorescent compound. In some embodiments, the multivalent molecule is a dimer of an immunoglobulin Fc fragment. In some embodiments, the multivalent molecule is a trimer of a trimerization domain molecule such as foldon.

In some embodiments, fluorescence-activated cell sorting is used to collect cells that remain bound to the labeled sorting reagent.

In some embodiments, the collected cells are sorted to single cells.

In some embodiments, nucleic acids encoding the antibody molecules are isolated from the single cells. In some embodiments, a host cell is transfected with a nucleic acid encoding an antibody heavy chain or variable domain thereof, and a nucleic acid encoding an antibody light chain or variable domain thereof; and the transfected cell may be grown under conditions to support expression of antibody by the transfected cell. In some embodiments, the host cell is Chinese hamster ovary (CHO) cell.

In some embodiments, the antibody producing cells are mammalian cells or yeast cells (such as S. cerevisiae or Pichia) that produce antibody molecules on the cell surface. In some embodiments, the antibody producing mammalian cells are primary antibody producing cells or immortalized cells lines which produce antibody molecules on the cell surface. In some embodiments, the primary antibody-producing cells are obtained from spleen, lymph node, peripheral blood and/or bone marrow of a mammal. In some embodiments, the primary antibody-producing cells are B cells. In some embodiments, the primary antibody-producing cells are obtained from a mouse immunized with an immunogen comprising a peptide-MHC trimer which comprises the target peptide and two polypeptide chains of the MHC molecule, wherein the two polypeptide chains comprises (i) an MHC class I α chain or a portion thereof and β2 microglobulin when the MHC molecule is a MHC class I molecule, or (ii) an MHC class II α chain or a portion thereof and an MHC class II β chain or a portion thereof when the MHC molecule is a MHC class II molecule, wherein the target peptide is presented in a peptide-binding groove formed by the two polypeptide chains of the MHC molecule. In some embodiments, the two polypeptide chains of the peptide-MHC trimer in the immunogen are identical in sequence to the two polypeptide chains of the peptide-MHC trimer in the sorting reagent. In some embodiments, the two polypeptide chains of the peptide-MHC trimer in the immunogen are identical in sequence to the two polypeptide chains of the peptide-MHC trimer in the blocking reagent and the two polypeptide chains in the peptide-MHC trimer(s) in the sorting reagent. In some embodiments, the immortalized cells lines are chosen from Chinese hamster ovary (CHO) cells, Human Embryonic Kidney (HEK) 293 cells, and hybridoma cells which produce antibody molecules on the cell surface.

### BRIEF DESCRIPTION OF THE DRAWINGS

The patent or application file contains at least one drawing executed in color. Copies of this patent or patent application publication with color drawings will be provided by the office upon request and payment of the necessary fee.

FIG. 1A is a schematic representation of exemplary MHC I/peptide trimer molecules, in single chain as shown here, used for PiG (Peptide-in-binding-Groove) antigen sorts and in-vitro binding experiments. In some exemplary embodiments, one cysteine residue can be engineered into the peptide linker and the tyrosine at position 108 in the alpha chain (Y108C) for disulfide stapling of the peptide.

FIG. 1B is a schematic illustration of antibody recognizing MHC I/peptide complex on the surface of a cell presenting a target peptide (e.g., a cancer cell presenting a peptide derived from a tumor associated antigen).

FIG. 2A schematically illustrates the direct antigen sort method. From left to right, the antibody-anchored cells are first contacted with a biotin labeled target peptide-ß2m-MHC trimer protein as a sorting reagent. Cells are washed to remove any unbound biotin labeled reagent and then contacted with PE labeled streptavidin. Cells expressing antibody molecules remaining bound to the biotin labeled sorting reagent are identified through PE labeled streptavidin. Obtained from this direct antigen sort method are cells expressing antibody molecules to various possible immunogenic epitopes of the sorting reagent target peptide-ß2m-MHC trimer protein.

FIG. 2B schematically illustrates the PiG antigen sort method that isolates cells expressing antibody molecules (in orange) that bind specifically to MHC/target peptide interface. Cells are first contacted with a block reagent containing an unlabeled control peptide-ß2m-MHC trimer protein. After pre-incubating with the unlabeled trimer blocking reagent, cells are further exposed to a biotin labeled sort reagent target peptide-ß2m-MHC trimer protein in the presence of the unlabeled trimer blocking reagent. The concentration of the blocking reagent in the preincubation step is in excess (e.g., 100 fold) of the concentration of the biotin labeled sorting reagent. Cells are washed to remove the unbound biotin labeled sort reagent and then contacted with PE labeled streptavidin. The cells expressing antibody molecules specifically to an MHC/target peptide interface remain bound to the biotin labeled sort reagent and are identified through PE labeled streptavidin.

FIGS. 3A-3B demonstrate the detection and separation of antibody-expressing B cells that express antibodies specific to MAGEA3 (108-116) (ALSRKVAEL, SEQ ID NO: 1)-ß2m-HLA-A2 using either the direct antigen sort method (3A) or the PiG sort method (3B). (3A) 61 B cells out of one million splenocytes expressing antibodies bind to the biotin labeled sort reagent were detected (yellow rectangle) from the mouse using the direct sort method. (3B) 9 B cells out of one million splenocytes expressing antibodies specific to the biotin labeled sort reagent were detected (yellow rectangle) using the PiG sort method (using MAGEA3 (271-279) (FLWGPRALV, SEQ ID NO: 2)-ß2m-HLA-A2 trimer protein as the blocking reagent). “mmh” stands for a Myc-Myc-His tag.

FIG. 4A demonstrates Luminex bindings of clones from the direct antigen sort method. Total three hundred and five clones were tested with biotin labeled MAGEA3 (108-116)-ß2m-HLA-A2 trimer protein, labeled as “on-target” binding signals, and with biotin labeled control peptide MAGEA3 (271-279)-ß2m-HLA-A2 trimer protein, labeled as “irrelevant” binding signals. Here, most of the clones were observed to align diagonally on the graph showing similar binding signals to both the “on-target” peptide and “irrelevant” control peptide-ß2m-HLA-A2 trimers. This trend indicates that these clones bind common epitopes shared between both the biotin labeled MAGEA3 (108-116)-ß2m-HLA-A2 trimer and the biotin labeled irrelevant peptide MAGEA3 (271-279)-ß2m-HLA-A2 trimer. There was only a very small subset of the clones (n=6 in this case) showing high “on-target” to “irrelevant” binding signal ratios, indicating specific “on-target” binding to the biotin labeled MAGEA3 (108-116)-ß2m-HLA-A2 trimer. These few clones may bind to the epitopes on the biotin labeled MAGEA3 (108-116)-ß2m-HLA-A2 trimer that the biotin labeled irrelevant peptide MAGEA3 (271-279)-ß2m-HLA-A2 trimer does not contain, and are called “On-Target Binders”. The unique epitopes of these on-target binders may reside in the specific HLA-A2/MAGEA3 (108-116) interface.

FIG. 4B demonstrates Luminex bindings of clones from the PiG antigen sort method. Total two hundred sixty-two clones were tested. Comparing to FIG. 4A, a lot fewer clones align diagonally on the graph showing similar binding signals to both the “on-target” and “irrelevant” peptide-ß2m-HLA-A2 trimers; instead, there was an enrichment of 22 fold of clones falling in the “On-Target Binders” gate showing high “on-target” to “irrelevant” binding ratios. These clones may bind to the unique epitopes on the biotin labeled MAGEA3 (108-116)-ß2m-HLA-A2 trimer that are not shared with the biotin labeled irrelevant peptide MAGEA3 (271-279)-ß2m-HLA-A2 trimer. This data showed specific MAGEA3 (108-116) Peptide-in-binding-Groove HLA-A2 complex interface (PiG) selectivity of antibodies isolated from the PiG sort method.

FIGS. 5A-5B demonstrate the detection and separation of antibody-producing B cells that express antibodies specific to MAGEA4 (230-239, GCYDGREHTV, SEQ ID NO: 3)-ß2m-HLA-A2 using either the direct antigen sort method (5A) or the PiG antigen sort method (5B). (5A) 353 B cells out of one million splenocytes were observed bound to the biotin labeled sort reagent (yellow rectangle) using direct sort method. (5B) 28 B cells out of one million splenocytes expressing antibodies specific to the biotin labeled sort reagent were detected (yellow rectangle) using PiG antigen sort method (using hLMP2 (aa426-434)(CLGGLLTMV, SEQ ID NO: 4)-ß2m-HLA-A2 trimer protein as the blocking reagent).

FIG. 6 demonstrates the Luminex binding assay results from the PiG antigen sort method. A series of protein-coupled beads tested in the immunoassay are listed in the X-axis in FIG. 6. Proteins coupled on beads annotated 68, 70, 19, 22, 25, and 60 are the “on-target” HLA-A2/ß2m trimer proteins with MAGEA4 (230-239) peptide, the rest are the HLA-A2/ß2m trimer proteins with “irrelevant” control peptides coupled on beads as annotated 67, 68, 18, 21, 78, 79, 50, 51, 52, 36, 37, 38, 39, 40, 41, 42, 43, 44, 45, 46, 47, 48, 49; or other MHC trimer proteins as annotated 81, 82, 83, 84, 89, 90; or different species beta2-microglobulin (ß2m) proteins as annotated 85, 86, 87; or irreverent control proteins as annotated 88, 23 and 24. Mean fluorescence intensities (MFI) measured below 1,000 are deemed non-specific bindings as denoted below the dotted line in the graph. Among the total 660 clones tested, the majority of the antibodies showed specific bindings (i.e. above 1,000 MFI) toward the six “on-target” HLA-A2/ß2m trimer proteins as colored in purple in the red rectangle, while comparably low number of antibodies show specific bindings toward the irreverent peptide HLA-A2/ß2m trimer proteins (clones either colored in light blue or grey) including MAGEA4 (286-294), NY-ESO (157-165), KRAS (both wild type and mutant peptides), MAGEA3 (271-279), HPV 16E7 (11-19), and HPV 16E7 (82-90), as well as to other MHC trimer proteins HLA-ß27, HLA-C, and mH2D(b), and to human ß2m, monkey ß2m, mouse ß2m proteins, as well as to several control proteins EGFP, IFNAR.hFc, and FelD1.mFc. These results indicate that the majority of the antibodies bind the epitopes residing in the unique “on-target” HLA-A2/ß2m/MAGEA4 (230-239) interfaces, and only a limited number of antibodies bind the common epitopes shared between the “on-target” HLA-A2/ß2m trimers and the irrelevant HLA-A2/ß2m trimers. The results showed specific MAGEA4 (230-239) Peptide-in-binding-Groove HLA-A2 complex interface (PiG) selectivity of antibodies isolated using the PiG sort method.

FIGS. 7A-7B demonstrate the detection and separation of antibody-producing B cells that express antibodies specific to DNTT (250-258)(KLFTSVFGV, SEQ ID NO: 5)-ß2m-HLA-A2 using either the direct antigen sort method (FIG. 7A) or the PiG antigen sort method (FIG. 7B). (7A) 363 B cells out of one million splenocytes were observed bound to the biotin labeled sort reagent (yellow rectangle) using direct sort method. (7B) 54 B cells out of one million splenocytes expressing antibodies specific to the biotin labeled sort reagent were detected (yellow rectangle) using PiG antigen sort method (using hPOLM (aa238-246)(KLFTQIFGV, SEQ ID NO: 6)-ß2m-HLA-A2 trimer protein and hPYGL (aa622-630)(KLITSVADV, SEQ ID NO: 7)-ß2m-HLA-A2 trimer protein as the blocking reagent).

FIG. 8 demonstrates the Luminex binding assay results from the PiG antigen sort method. A series of protein-coupled beads tested in the immunoassay are listed in the X-axis in FIG. 8. Proteins coupled on beads annotated 13, 14, and 62 are the “on-target” DNTT (250-258) peptide-ß2m-HLA-A2 trimer protein, the rest are the HLA-A2/ß2m trimer proteins with either the “off-target” peptides hPOLM (aa238-246) (KLFTQIFGV, SEQ ID NO: 6) and hPYGL (aa622-630) (KLITSVADV, SEQ ID NO: 7) coupled on beads as annotated 7 and 8 respectively, or the “irrelevant” peptides coupled on beads as annotated 15, 16, 63, 9, 10, 20, 23, 65, 42, 54, 24, 27, 47, 28, 29, 48, 30, 31, 35, 49, 52; or other MHC trimer proteins as annotated 45, 53, 32, 50, 33, 34, 36, 51; or different species beta2-microglobulin (ß2m) proteins as annotated 37, 38, 41; or irreverent control proteins as annotated 43, 44, 5, 3, 4, 2, 1, and 66. Mean fluorescence intensities (MFI) measured below 1,000 are deemed non-specific bindings as denoted below the dotted line in the graph. Among the total 156 clones tested, the majority of the antibodies showed specific bindings (i.e. above 1,000 MFI) toward the two “on-target” HLA-A2/ß2m trimer proteins as colored in blue in the “on-target” rectangle, while showing no bindings toward the two “off-target” peptide HLA-A2/ß2m trimer proteins (hPOLM (aa238-246) and hPYGL (aa622-630)) as colored in orange, and showing comparably low or no bindings toward the rest of the proteins (colored in grey) including irreverent peptide HLA-A2/ß2m trimer proteins DNTT (475-483), ACTR10 (335-343), DSCAML1 (402-410), NY-ESO (157-165), MAGEA3 (271-279), HPV 16E7 (11-19), EBNA1 (562-570), LMP2 (426-434), and LMP2 (329-337), as well as to other MHC trimer proteins HLA-A29, HLA-ß27, HLA-C, and mH2D(b), to human ß2m, monkey ß2m, mouse ß2m proteins, and to several control proteins EGFP, TL1A.6His, IL6Ra, and FelD1. This indicating that the majority of the antibodies specifically bind the epitopes resides in the unique “on-target” DNTT (250-258)-HLA-A2 interfaces, and only a handful of antibodies bind the common epitopes shared between the “on-target” peptide)-ß2m-HLA-A2 trimers and the irrelevant peptide)-ß2m-HLA-A2 trimers. The results showed specific DNTT (250-258) Peptide-in-binding-Groove HLA-A2 complex interface (PiG) selectivity of antibodies isolated from the PiG sort method.

FIG. 9A schematically illustrates the direct antigen sort method as depicted in FIG. 2A.

FIG. 9B schematically illustrates a 2-color PiG antigen sort method that allows the identification and isolation of cells expressing antibodies (in orange) that bind specifically to MHC/target peptide interface from cells expressing antibodies (in purple) that bind to both MHC/target peptide and MHC/control peptide. Cells are contacted with a labeled block reagent containing an Alexa Fluor 647 conjugated control peptide-ß2m-MHC trimer protein, and a labeled sorting reagent containing a biotin labeled target peptide-ß2m-MHC trimer protein. Cells are then washed to remove any unbound reagents and then contacted with PE labeled streptavidin. The concentration of the labeled blocking reagent can be similar to or higher than the concentration of the labeled sorting reagent. Cells expressing antibodies specifically to an MHC/target peptide interface are bound only to the biotin labeled sort reagent and are identified through PE labeled streptavidin. Cells expressing antibodies against other epitopes of MHC/target peptide are bound to both the Alexa Fluor 647 labeled blocking reagent and the biotin labeled sorting reagent, thus stained with both Alexa Fluor 647 and PE though PE labeled streptavidin. Here, cells expressing antibodies specifically to an MHC/target peptide interface are then separated from the rest through differential staining profiles.

FIG. 10A is a schematic representation of exemplary MHC class II/peptide trimer molecules used for PiG (Peptide-in-binding-Groove) antigen sorts and in-vitro binding experiments. The MHC class II α chain or the portion thereof comprises the α1 domain and the α2 domain but does not comprise a transmembrane domain or a cytoplasmic domain, the MHC class II β chain or the portion thereof comprises the β1 domain and the β2 domain but does not comprise a transmembrane domain or a cytoplasmic domain, and the MHC class II α chain or the portion thereof and the MHC class II β chain or the portion thereof are linked by a Jun-Fos zipper comprising a Jun leucine zipper dimerization motif and a Fos leucine zipper dimerization motif. In some exemplary embodiments, a cysteine residue can be engineered into the peptide linker and the Arginine at position 101 in the α chain (R101C) for disulfide stapling of the peptide.

FIGS. 10B-10C demonstrate the detection and separation of antibody-producing B cells that express antibodies specific to Gliadin (QLQPFPQPELPY, SEQ ID NO: 8)-HLA-DQB.Jun-HLA-DQA.Fos trimer using either the direct antigen sort method (FIG. 10B) or the PiG antigen sort method (FIG. 10C). (10B) 165 B cells out of one million splenocytes were observed bound to the biotin labeled sort reagent (yellow rectangle) using direct sort method. (10C) 92 B cells out of one million splenocytes expressing antibodies specific to the biotin labeled sort reagent were detected (yellow rectangle) using PiG antigen sort method using HSV (EEVDMTPADALD, SEQ ID NO: 9)-HLA-DQB.Jun.PADRE.LCMV.HLA-DQA.Fos trimer as the blocking reagent.

FIG. 10D demonstrates the Luminex binding assay results from the PiG antigen sort method. A series of protein-coupled beads tested in the immunoassay are listed in the X-axis in FIG. 10D. Proteins coupled on beads annotated 11, 9, 13, 12, 15, 13, and 43 are the “on-target” HLA-DQA/HLA-DQB trimer proteins with Gliadin peptide, the rest are the HLA-DQA/HLA-DQB trimer proteins with “irrelevant” control peptides coupled on beads as annotated 18, 20, 21, 42, 2, 3, 4, 44 and 7; or other MHC trimer proteins as annotated 36 and 38; or irrelevant control proteins as annotated 1, 27 and 28. Mean fluorescence intensities (MFI) measured below 1,000 are deemed non-specific bindings as denoted below the dotted line in the graph. Among the total 88 clones tested, the majority of the antibodies showed specific bindings (i.e. above 1,000 MFI) toward the seven “on-target” HLA-DQA/HLA-DQB trimer proteins in the red rectangle, as shown in FIG. 10D, from left to right, with 79 clones, 83 clones, 79 clones, 82 clones, 73 clones, 74 clones, and 84, respectively, out of total 88 clones, while comparably lower numbers of antibodies show specific bindings toward the irrelevant peptide HLA-DQA/HLA-DQB trimer proteins including other Gliadin peptides PQPELPYPQPQL (SEQ ID NO: 10) and FPQPEQPFPWQP (SEQ ID NO: 11), as well as to other MHC trimer proteins HLA-A2/ß2m, as well as to several control proteins hFelD1.mmh, IL6Ra.mmh and Bt-IL6Ra.mmh. These results indicate that the majority of the antibodies bind the epitopes residing in the unique “on-target” HLA-DQA/HLA-DQB/Gliadin (QLQPFPQPELPY, SEQ ID NO: 8) interfaces, and only a limited number of antibodies bind the common epitopes shared between the “on-target” HLA-DQA/HLA-DQB trimers and the irrelevant HLA-DQA/HLA-DQB trimers. The results showed specific Gliadin (QLQPFPQPELPY, SEQ ID NO: 8) Peptide-in-binding-Groove HLA-DQ complex interface (PiG) selectivity of antibodies isolated using the PiG sort method.

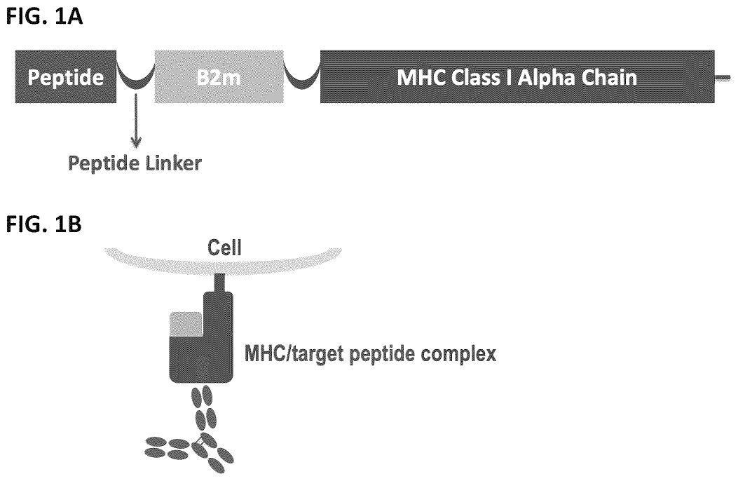

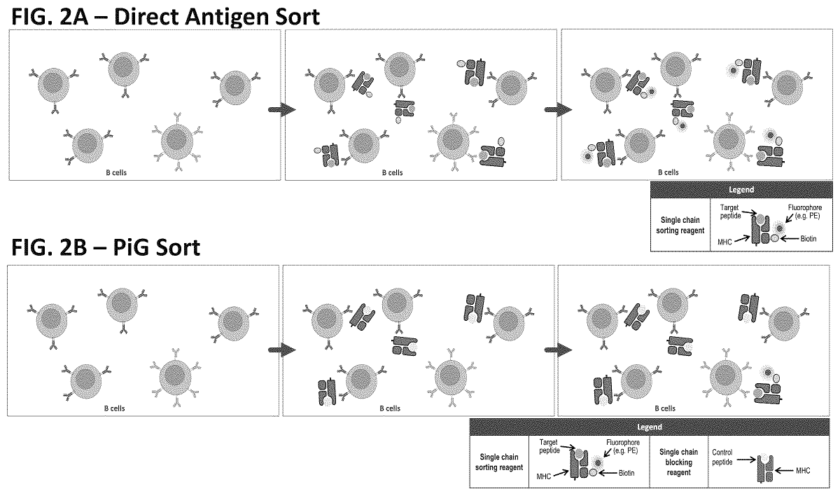

### DETAILED DESCRIPTION

Although claimed subject matter will be described in terms of certain examples, other examples, including examples that do not provide all the benefits and features set forth herein, are also within the scope of this disclosure. Various structural, logical, and process step changes may be made without departing from the scope of the disclosure. Features described herein in one embodiment or some embodiments can be combined with features described herein in another or other embodiments unless it is apparent that the features are mutually exclusive.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value).

In the description that follows, certain conventions will be followed as regards to the usage of terminology. Generally, terms used herein are intended to be interpreted consistently with the meaning of those terms as they are known to those of skill in the art. In practicing the methods described herein, many conventional techniques in molecular biology, microbiology, cell biology, biochemistry, and immunology are used, which are within the skill of the art. These techniques are described in greater detail in, for example, Molecular Cloning: a Laboratory Manual 4th edition, J. F. Sambrook and D. W. Russell, ed. Cold Spring Harbor Laboratory Press 2012; Recombinant Antibodies for Immunotherapy, Melvyn Little, ed. Cambridge University Press 2009; “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., ed., 1994); “A Practical Guide to Molecular Cloning” (Perbal Bernard V., 1988); “Phage Display: A Laboratory Manual” (Barbas et al., 2001). The contents of these references and other references containing standard protocols, widely known to and relied upon by those of skill in the art, including manufacturers' instructions are hereby incorporated by reference as part of the disclosure.

### General Description

It has been discovered herein that inclusion of a blocking step when sorting antibody-producing cells permits enrichment of cells expressing rare antibody molecules that specifically recognize an MHC-peptide interface. For example, to isolate cells that express antibody molecules specific for an interface between a target peptide and an MHC molecule from a population of antibody-producing cells encompassing cells expressing on the cell surface antibody molecules specific for an interface between the target peptide and an MHC molecule, the cell population can be first incubated with a blocking reagent comprising a peptide-MHC trimer which comprises a control peptide and two polypeptide chains of the MHC molecule, with the control peptide being presented in a peptide-binding groove formed by the two polypeptide chains of the MHC molecule. Subsequently, the cell population are contacted with a sorting reagent in the presence of the blocking reagent, wherein the sorting reagent comprises a labeled peptide-MHC trimer comprising the target peptide and two polypeptide chains of the MHC molecule, with the target peptide being presented in a peptide-binding groove formed by the two polypeptide chains of the MHC molecule. After washing the cells to remove unbound sorting reagent, cells that remain bound to the sorting reagent can be collected to obtain cells expressing antibody molecules specific for an interface between the target peptide and the MHC molecule. The methods disclosed herein permits a significant enrichment of cells expressing antibody molecules that specifically recognize an MHC-peptide interface as compared to without using a blocking reagent.

The term “antibody molecule”, as used herein, encompasses both full length antibodies and antigen-binding fragments thereof (e.g., Fab, Fab′, and F(ab′)₂).

The term “enriching” as used herein means increasing the frequency or percentage of desired cells in a cell population, e.g., increasing the percentage of antibody-producing cells expressing antibody molecules specific for a peptide-MHC interface within a cell population containing cells expressing antibody molecules specific for a peptide-MHC interface and cells expressing antibody molecules not specific for a peptide-MHC interface (such as antibody molecules directed against other epitopes of the MHC molecule not involved in a peptide-MHC interface). In this disclosure, a blocking reagent is utilized that comprises one or more control peptide-MHC trimers, and the present method includes an initial blocking step by incubating the cells with a blocking reagent, followed by a sorting step by incubating the cells with a sorting reagent comprising a target peptide-MHC trimer in the presence of the blocking reagent. Cells remaining bound to the sorting reagent are thereafter collected and are enriched in cells expressing antibody molecules specific for an MHC-peptide interface. That is, the cell population obtained as a result of the disclosed method is enriched in antibody-producing cells expressing antibody molecules specific to a peptide-MHC interface. The extent of enrichment is at least 2 fold-30 fold, i.e., at least 2 fold, at least 3 fold, at least 4 fold, at least 5 fold, at least 10 fold, at least 15 fold, at least 20 fold, at least 25 fold, at least 30 fold, or greater, as measured based on the percentage of cells expressing an antibody specific to a peptide-MHC interface in a population of cells obtained after performing the methods disclosed herein using a blocking reagent as compared to without using a blocking reagent. In some embodiments, at least 40% (e.g., 40%, 50%, 60%, 70%, 80% or more) of the cells in the cell population obtained are cells expressing antibody molecules specific to a peptide-MHC interface.

Antibody molecules specific to a peptide-MHC interface include antibody molecules that exhibit a higher binding affinity to the peptide presented in the context of the MHC molecule, as compared to a control peptide presented in the context of the MHC molecule. For instance, peptide-MHC trimers described herein can be utilized in assessing antibody specificity. In some embodiments, antibody molecules specific to an interface between a target peptide and an MHC molecule exhibit a higher binding affinity to a peptide-MHC trimer wherein the target peptide is presented in a peptide-binding groove formed by two polypeptide chains of the MHC molecule, as compared to a peptide-MHC trimer wherein a control peptide is presented in the peptide-binding groove formed by the same two polypeptide chains of the MHC molecule. In some embodiments, the binding affinity for a target peptide-MHC trimer is at least 10 or 20 times higher than for a control peptide-MHC trimer. In some embodiments, the binding affinity for a target peptide-MHC trimer is at least 30 to 50 times higher than for a control peptide-MHC trimer. In some embodiments, the binding affinity for a target peptide-MHC trimer is at least 50-100 times higher than for a control peptide-MHC trimer. In some embodiments, antibody molecules that exhibit binding to a target peptide-MHC trimer at a level below a threshold value are considered to be antibody molecules non-specific to an interface between the target peptide-MHC trimer. For example, antibody molecules that exhibit binding to a target peptide-MHC trimer at a level below a Mean fluorescence intensity (MFI) measurement of 1000 in a Luminex assay are considered to be antibody molecules non-specific to an interface between the target peptide-MHC trimer. In some embodiments, antibody molecules that exhibit binding to a target peptide-MHC trimer at a level above a threshold value and exhibit binding to one or more control peptide-MHC trimers below the threshold level are considered to be antibody molecules specific to an interface between the target peptide-MHC trimer. For example, antibody molecules that exhibit binding to a target peptide-MHC trimer at a level above MFI of 1000 in a Luminex assay and exhibit binding to one or more control peptide-MHC trimers below MFI of 1000 are considered to be antibody molecules specific to an interface between the target peptide-MHC trimer.

### MHC Molecules

The terms “major histocompatibility complex” and “MHC” encompass the terms “human leukocyte antigen” or “HLA” (the latter two of which are generally reserved for human MHC molecules), naturally occurring MHC molecules, functional mutants and derivatives, wherein such functional mutants and derivatives retain the ability to display an antigenic peptide for recognition by a T-cell receptor (TCR), e.g., an antigen-specific TCR. Examples of functional mutant MHC molecules include those comprising substitution or addition of amino acids to accommodate a disulfide bridge with a peptide to be presented or a peptide linker, as described herein.

MHC molecules are generally classified into two categories: class I and class II MHC molecules. MHC class I molecules are heterodimers composed of two polypeptide chains, a (heavy) chain (an integral membrane protein) and β₂-microglobulin (B2M)(invariable light chain, a soluble protein). The α chain includes an extracellular portion (ectodomain) comprising αl, α2 and α3 domains, a transmembrane domain and a cytoplasmic tail. In naturally occurring MHC class I molecules, the two polypeptide chains are non-covalently associated with each other via interaction between β₂-microglobulin and the as domain of the α chain. The αl and α2 domains of the α chain form a peptide binding groove that can bind a peptide typically of about 8-10 amino acids. MHC class II molecules are also heterodimers composed of two polypeptide chains: α chain and β chain. Both α chain and β chain include an extracellular portion (ectodomain), a transmembrane domain and a cytoplasmic tail. The extracellular portion of the α chain comprises αl and α2 domains, and the extracellular portion of the β chain comprises β1 and β2 domains. The α1 and β1 domains come together to form a peptide-binding domain groove that binds a peptide of generally between 15 and 24 amino acids in length.

In some embodiments, the MHC molecule used herein may be a human HLA molecule. In some embodiments, the MHC class I or MHC class II polypeptides may be derived from any functional human HLA-A, B, C, DR, or DQ molecules. In some embodiments, the MHC class I molecule may be selected from HLA-A, HLA-B, HLA-C, or HLA-E, such as HLA-A*02, HLA-A*01, HLA-A*03, HLA-A*11, HLA-A*23, HLA-A*24, HLA-B*07, HLA-B*08, HLA-B*40, HLA-B*44, HLA-B*15, HLA-C*04, HLA*C*03, and HLA-C*07. In some embodiments, a human class II MHC molecule is selected from the group consisting of HLA-DP, HLA-DR, HLA-DQ, HLA-DM and HLA-DO.

### Peptide-MHC Trimer

The term “peptide-MHC trimer” refers to a trimeric entity that comprises a peptide and two polypeptide chains of an MHC molecule, wherein the two polypeptide chains are either (i) the α chain or a portion thereof of the MHC molecule and β2 microglobulin or a portion thereof when the MHC molecule is an MHC class I molecule, or (ii) the α chain or a portion thereof and the β chain or a portion thereof of the MHC molecule when the MHC molecule is an MHC class II molecule; and wherein the peptide is presented in a peptide-binding groove formed by the two polypeptide chains, such that the trimer can specifically bind an antigen recognition molecule such as a T-cell receptor.

In some embodiments, the peptide is not covalently linked to either of the two MHC polypeptide chains and is bound to the two MHC polypeptide chains through non-covalent interactions to be presented in a peptide-binding groove formed by the two MHC polypeptide chains. A non-covalently associated peptide-MHC trimer can be prepared using conventional methodologies. An exemplary method is described in the Examples section below.

In some embodiments, the peptide is covalently linked to at least one of the two MHC polypeptide chains. In some embodiments, the covalent linkage is accomplished through a peptide linker, as further described below. In some embodiments, the covalent linkage is accomplished through a disulfide bond formed between a cysteine residue in the peptide or a peptide linker and a cysteine residue in one of the two MHC polypeptide chains, as further described below.

In some embodiments, the 3 components of the trimer, i.e., the peptide and the two polypeptide chains are linked in a single polypeptide chain to form a single chain trimer. Examples of a single chain peptide-MHC trimer include a single chain peptide-MHC class I trimer comprising a peptide, β2 microglobulin or a portion thereof, and MHC class I α chain or a portion thereof, and a single chain peptide-MHC class II trimer comprising a peptide, a β chain or a portion thereof of an MHC class II molecule, an α chain or a portion thereof of the MHC class II molecule. The covalent linkage between the components of the trimer can be achieved by a peptide linker. In some embodiments, a single chain peptide-MHC class I trimer comprises, from N- to C-terminus, peptide-β2 microglobulin or a portion thereof-α chain or a portion thereof of an MHC class I molecule (wherein the hyphen “-” represents a peptide linker), such as those described in e.g., U.S. Pat. No. 8,895,020B2, U.S. Pat. No. 8,992,937; Hansen et al. (2010) Trends Immunol. 31:363-69; Truscott et al. (2007) J. Immunol. 178:6280-89; Mitaksov et al. (2007) Chem Biol 14:909-22, Lybarger et al., JBC 278 (29): 27105-27111 (2003), each of which is incorporated herein by reference. In some embodiments, the β2 microglobulin sequence can comprise a full-length (human or non-human) β2 microglobulin sequence. In certain embodiments, the β2 microglobulin sequence lacks the leader peptide sequence (i.e., the mature form). As such, the β2 microglobulin sequence can comprise about 99 amino acids, and can be a human β2 microglobulin sequence (Genebank AF072097.1). A single-chain trimer can further comprise a signal peptide sequence at the amino terminus. In some embodiments, a single-chain molecule can also comprise a disulfide bridge, as further discussed below.

In some embodiments, the peptide is covalently linked to one of the two MHC polypeptide chains, and noncovalent association with the other MHC polypeptide chain is accomplished by attaching (covalently linking) a binding pair of peptides to the two MHC polypeptide chains, respectively. Binding pairs of peptides, i.e., pairs of peptides that bind to each other, include, for example, a Jun-Fos zipper and knobs-into-holes. For instance, a Jun-Fos zipper can comprise a Jun leucine zipper dimerization motif (e.g., SEQ ID NO: 22) and a Fos leucine zipper dimerization motif (e.g., SEQ ID NO: 23). In some embodiments, one of the two MHC polypeptide chains in the trimer is attached (i.e., covalently linked), e.g., through a peptide linker, to a Jun leucine zipper dimerization motif, and the other MHC polypeptide chain is covalently linked, e.g., through a peptide linker, to a Fos leucine zipper dimerization motif. In some embodiments, a Jun leucine zipper dimerization motif and a Fos leucine zipper dimerization motif are attached to the C-terminus of the two MHC polypeptide chains, respectively. In some embodiments, a peptide-MHC trimer is a peptide-MHC class II trimer comprising, (i) from N- to C-terminus, a peptide-α chain or a portion thereof of an MHC class II molecule-one of a Jun leucine zipper dimerization motif and a fos leucine zipper dimerization motif, and (ii) from N- to C-terminus: β chain or a portion thereof of the MHC class II molecule-the other one of a Jun leucine zipper dimerization motif and a fos leucine zipper dimerization motif (wherein the hyphen “-” represents a peptide linker), such as those described in WO2021/113297A1 (Regeneron Pharmaceuticals, Inc.), incorporated herein by reference. In some embodiments, a peptide-MHC trimer is a peptide-MHC class II trimer comprising, (i) from N- to C-terminus, a peptide-β chain or a portion thereof of an MHC class II molecule-one of a Jun leucine zipper dimerization motif and a fos leucine zipper dimerization motif, and (ii) from N- to C-terminus: α chain or a portion thereof of the MHC class II molecule-the other one of a Jun leucine zipper dimerization motif a fos leucine zipper dimerization motif (wherein the hyphen “-” represents a peptide linker), such as those described in WO2021/113297A1 (Regeneron Pharmaceuticals, Inc.), incorporated herein by reference.

The term “portion” as used herein in referring to an MHC polypeptide chain includes at least a fragment of an MHC molecule required for forming a peptide binding groove. In some embodiments, a portion of an MHC class I α chain includes a fragment of the extracellular sequence (ectodomain) of MHC class I α chain required forming a peptide binding groove, e.g., a fragment of the extracellular sequence that comprises portions of the αl and α2 domains of the α chain capable of forming two β-pleated sheets and two α helices, or a fragment of the extracellular sequence that comprises the αl and α2 domains of the α chain. Inclusion of a portion of the β2 microglobulin chain stabilizes the MHC class I molecule. In some embodiments, a portion of an MHC class II α chain includes a fragment of the extracellular sequence (ectodomain) of the MHC class II α chain required for forming a peptide binding groove, e.g., a fragment of the extracellular sequence that comprises portions of the α1 domain capable of forming a β-pleated sheet and an α helix, a fragment of the extracellular sequence that comprises the α1 domain, a fragment of the extracellular sequence that comprises α1 and α2 domains, or the entire extracellular sequence of the α chain. In some embodiments, a portion of an MHC class II β chain includes a fragment of the extracellular sequence (ectodomain) of MHC class II β chain required for forming a peptide binding groove, e.g., a fragment of the extracellular sequence that comprises portions of the β1 domain capable of forming a β-pleated sheet and an α helix, a fragment of the extracellular sequence that comprises the β1 domain, a fragment of the extracellular sequence that comprises β1 and β2 domains, or the entire extracellular sequence of the β chain.

In some embodiments, the peptide-MHC trimer is soluble (i.e., not membrane bound), and the MHC polypeptide chains do not comprise a transmembrane domain or do not comprise transmembrane and cytoplasmic domains.

### Peptide Linker

A peptide linker can be designed to allow a peptide to fold into the binding groove of the MHC class molecule, resulting in a functional peptide-MHC trimer, e.g., a peptide-MHC trimer that binds TCR. Suitable size and sequences of linkers can be determined by using known methodologies, including conventional computer modeling techniques based on the predicted tertiary structure of a peptide-MHC trimer. As a non-limiting example, a linker can be about 9 amino acids to about 50 amino acids in length. In some embodiments, a linker can be at least about 9 amino acids, at least about 10 amino acids, at least about 11 amino acids, at least about 12 amino acids, at least about 13 amino acids, at least about 14 amino acids, or at least about 15 amino acids in length. In some embodiments, a linker can be no more than about 50 amino acids, no more than about 45 amino acids, no more than about 40 amino acids, no more than about 35 amino acids, no more than about 30 amino acids, no more than about 25 amino acids, no more than about 20 amino acids, or no more than about 15 amino acids in length. Any suitable amino acids can be used in a linker. In some embodiments, non-limiting examples of suitable linkers including flexible linkers, rigid linkers, and cleavable linkers are reviewed, for example, in Chen et al. (2013) Adv. Drug Deliv. Rev. 65(10):1357-1369, herein incorporated by reference in its entirety for all purposes. Linkers used in the peptide-MHC trimers disclosed in some embodiments herein can have one or more or all of the following features: the linkers are flexible, the linkers are non-immunogenic, the linkers do not include charged amino acids, the linkers include polar amino acids, and any combination thereof. Flexibility can be achieved, for example, by using linkers rich in small or hydrophilic amino acids (e.g., but not limited to, glycine and serine). Including polar amino acids can help improve solubility. In some embodiments, non-limiting examples of polar amino acids include Asn, Gln, Ser, and Thr. Omitting charged amino acids (e.g., but not limited to, Lys, Arg, Glu, and Asp) can help avoid electrostatic interactions with other amino acid side chains. Some linkers suitable for use in the disclosed peptide-MHC trimers predominantly comprise amino acids with small side chains, such as glycine, alanine, and serine (e.g., but not limited to, glycine and serine). In some embodiments, exemplary linkers include glycine polymers (G)n, glycine-serine polymers such as (GGGGS)ₙ (SEQ ID NO: 12), where n is an integer of at least one, glycine-alanine polymers, alanine-serine polymers, and other flexible linkers that are well-known.

### Disulfide Bridge

In some embodiments of peptide-MHC trimers disclosed herein, the peptide or a linker connecting the peptide to one of the two MHC polypeptide chains in the trimer is attached via a disulfide bridge, i.e., a disulfide bond between a cysteine residue in the peptide or the linker and a cysteine residue in one of the two MHC polypeptide chains of the trimer. Such a disulfide bond can help position the peptide in the peptide-binding groove of the MHC molecule. See, e.g., U.S. Pat. Nos. 8,992,937, 8,895,020, and WO2021/113297A1 (Regeneron Pharmaceuticals, Inc.), each of which is incorporated in its entirety by reference.

In some embodiments, the MHC polypeptide chain comprises only one cysteine and no other cysteines or no other unpaired cysteines (for example, no other Cys being able to form a disulfide bond within the MHC polypeptide chain). The cysteine in an MHC polypeptide chain of a trimer can be a naturally occurring cysteine (i.e., present in an unmodified (i.e., wild type) MHC polypeptide chain) or it can be a mutation (addition or substitution) relative to the unmodified (i.e., wild type) MHC polypeptide chain.

In some embodiments, the disulfide bond comprises a first cysteine, that is positioned within a linker extending from the carboxy terminal of a peptide, and a second cysteine that is positioned within an MHC class I α chain. In certain embodiments, the second cysteine can be a mutation (addition or substitution) in the MHC class I α chain. In some embodiments, the peptide-MHC trimer is a single-chain molecule comprising from N-terminus to C-terminus: a peptide, a first linker that comprises a first cysteine, a β2-microglobulin sequence or a portion thereof, a second linker, and an MHC class I α chain or a portion thereof comprising a second cysteine, wherein the first cysteine and the second cysteine form a disulfide bridge. In some embodiments, the second cysteine is a substitution of an amino acid in a native MHC class I α chain selected from the group consisting of T80C, Y84C and N86C. Y84C refers to a mutation at position 108 in a mature form of MHC class I α chain, where the mature protein lacks a signal sequence. Alternatively, when the protein still includes a 24-mer signal sequence, the substitution is instead referred to as Y108C.

In some embodiments, a disulfide bond comprises a first cysteine, that is positioned within a linker extending from the carboxy terminal of a peptide, and a second cysteine that is positioned within an MHC class II polypeptide chain. In certain embodiments, the second cysteine can be a mutation (addition or substitution) in the MHC class II polypeptide chain. In some embodiments, position 101 in an MHC class II α chain sequence can be mutated to cysteine (R101C). As another embodiment, position 79 in an MHC class II α chain sequence (e.g., HLA class II histocompatibility antigen, DR alpha chain; NCBI Accession No. P01903.1) can be mutated to cysteine (F79C). See, e.g., WO2021/113297A1 (Regeneron Pharmaceuticals, Inc.), incorporated herein by reference. In some embodiments of peptide-MHC class II complexes, a residue that is a cysteine in an unmodified (i.e., wild type) α chain (i.e., the MHC class II α chain or portion thereof) of the MHC class II molecule or an unmodified (i.e., wild type) β chain (i.e., the MHC class II β chain or portion thereof) of the MHC class II molecule can be mutated to a non-cysteine residue to minimize disulfide scrambling (i.e., disulfide bonds forming between cysteine residues other than those intended to be used). The amino acid that is substituted for the cysteine can be chosen to allow proper folding of the MHC complex. In one exemplary embodiment, the cysteine is mutated to alanine because it has a minimal side chain and is therefore least disruptive sterically. In one exemplary embodiment, the cysteine at position 70 in the MHC class II α chain sequence set forth in SEQ ID NO: 49 (HLA class II histocompatibility antigen, DQ alpha 1 chain; NCBI Accession No. P01909.1) can be mutated, e.g., to alanine (C70A). See, e.g., WO2021/113297A1 (Regeneron Pharmaceuticals, Inc.), incorporated herein by reference.

### Other Components

In some embodiments, a peptide-MHC trimer described herein can also comprise one or more other peptide sequences, e.g., peptide sequences capable of stimulating T helper cells or capable of boosting an immune response. Such T helper cell epitopes or immunostimulatory peptides may be linked (e.g., but not limited to, covalently linked) to the peptide-MHC class I trimer. In an exemplary embodiment, such T helper cell epitope or immunostimulatory peptide to the C-terminus of a polypeptide chain of an MHC molecule described herein, e.g., but not limited to, an MHC class II α chain or portion thereof, an MHC class II β chain or portion or fragment, or an MHC class I α chain or portion thereof. Covalent linkage can be direct or via a linker such as a peptide linker.

As a non-limiting example of some embodiments, a T helper cell epitope suitable for use herein is a pan-DR-binding epitope (PADRE) peptide, which is a “pan-DR-binding epitope” that is a mouse MHC-II binding sequence used to boost the immune response based on providing a “universal” MHC-II epitope that is presented by mouse MHC I-Ab haplotype as described in Alexander et al. (2000) J. Immunol. 164(3):1625-1633, herein incorporated by reference in its entirety for all purposes. It can be fused the termini of antigen used in the immunization, and its uptake by antigen-presenting cells and presentation by MHC-II improves overall immune response. See, e.g., U.S. Pat. Nos. 6,413,935; 5,736,142; and Alexander et al. (1994) Immunity 1(9):751-761, each of which is incorporated herein by reference in its entirety for all purposes. These peptides have been shown to help in the generation of various immune responses against antigens. In some embodiments, the PADRE peptide can comprise or consist of AKFVAAWTLKAAA (SEQ ID NO: 13).

Like PADRE, peptides from lymphocytic choriomeningitis virus (LCMV) (e.g., but not limited to, from LCMV glycoprotein (GP), nucleoprotein (NP), or zinc-binding protein (Z)) are an alternate MHC-II binding small polypeptide that can be used to boost immune response. In some embodiments, such peptides can be used in addition to or as an alternative to PADRE. In some embodiments, the LCMV peptide used can be an LCMV-specific MHC class II-restricted CD4+ T cell epitope. See, e.g., Botten et al. (2010) Microbiol. Mol. Biol. Rev. 74(2):157-170 and Mothe et al. (2007) J. Immunol. 179(2):1058-1067, each of which is herein incorporated by reference in its entirety for all purposes. In some embodiments, the peptide can comprise or consist of SERPQASGVYMGNLT (SEQ ID NO: 14).

In some embodiments, peptides or other tags can also be included in a peptide-MHC trimer to facilitate, for example, purification. In some embodiments, non-limiting examples of tags suitable for use herein include, but are not limited to, E. coli biotin ligase (BirA), myc-myc-histidine (mmH), glutathione-s-transferase (GST), maltose binding protein (MBP), chitin binding protein (CBP), FLAG, and 1D4 (i.e., the 9 amino acid 1D4 epitope derived from the C-terminus of bovine rhodopsin). In some embodiments, the sequence of the BirA tag can comprise or consists of GLNDIFEAQKIEWHE (SEQ ID NO: 15). In some embodiments, the sequence of the mmH tag can comprise or consist of EQKLISEEDLEQKLISEEDLHHHHHH (SEQ ID NO: 16) or EQKLISEEDLGGEQKLISEEDLHHHHHH (SEQ ID NO: 17).

### Target Peptide and Sorting Reagent

The methods disclosed herein utilizes a sorting reagent that comprises a peptide-MHC trimer wherein the peptide in the MHC trimer is a target peptide.

The term “target peptide” as used herein refers to a peptide that binds to a peptide-binding grove of an MHC molecule, and it is the antibody molecules specifically directed to an interface between the target peptide and the MHC molecule that are being sought by the methods disclosed herein. Target peptides suitable for use in a peptide-MHC trimer disclosed herein include peptides that are derived from or comprise a fragment of autoantigens, tumor-associated antigens, infectious agents, toxins, allergens, or combinations thereof.

In some embodiments, a target peptide comprises at least a fragment of a human self-protein associated with an autoimmune disorder. In some embodiments, a target peptide comprises a fragment of an allergen. In some embodiments, a target peptide is associated with a T-cell-mediated disease (e.g., but not limited to, T-cell-mediated autoimmune disease such as type 1 diabetes mellitus, rheumatoid arthritis, multiple sclerosis, celiac disease, Addison's disease, and hypothyroidism). In some embodiments, a target peptide comprises a fragment of a protein of an infectious agent (e.g., but not limited to, bacterial, viral, or parasitic organisms). Examples of a target peptide comprising a fragment of a viral protein include Hepatitis B virus polymerase peptide POL606-616 (GSLPQEHIVQK, SEQ ID NO: 18), HIV GAG peptide AISPRTLNA (SEQ ID NO: 19), or Epstein-Barr virus peptide BZLF1 (40-48) (SQAPLPCVL, SEQ ID NO: 20). In some embodiments, a target peptide comprises a fragment of a tumor-associated protein. In some embodiments, tumor-associated antigens include a mutated oncogene such as a KRAS neoantigens, a viral oncogene such as HPV E7, a lineage differentiation antigen (such as melanocyte antigens like gp100), an oncofetal antigen such as WT-1, and a cancer-testis antigen (CTA) such as a MAGE family member. In some embodiments, a target peptide is a peptide fragment of MAGE-A3 or MAGE-A4, hMAGE-A3 aa108-116 (ALSRKVAEL, SEQ ID NO: 1), hMAGEA4 aa230-239 (GVYDGREHTV₂₃₀₋₂₃₉, SEQ ID NO: 3) or hMAGE-A4 aa 286-294 (KVLEHVVRV₂₈₆₋₂₉₄, SEQ ID NO: 21). In some embodiments, a target peptide is a peptide fragment of DNA nucleotidylexotransferase (DNTT), DNTT aa250-258 (KLFTSVFGV, SEQ ID NO: 5).

In some embodiments, peptides can be any suitable length for binding to an MHC protein in a manner such that the peptide-MHC trimer can bind to a TCR and e.g., effect a T cell response. The length of peptides can vary, for example, from about 5 to about 40 amino acids (e.g., but not limited to, from about 6 to about 30 amino acids, from about 8 to about 25 amino acids, from about 10 to about 20 amino acids, from about 12 to about 18 amino acids, or any size peptide between 5 and 40 amino acids in length, in whole integer increments (i.e., 5, 6, 7, 8, 9 . . . 40)). While naturally MHC-class-II-bound peptides vary from about 9 to about 40 amino acids, in nearly all cases the peptide can be truncated to a 9-11 amino acid core without loss of MHC-binding activity or T-cell recognition. In some embodiments, the MHC II peptide can be from 9 to 25 amino acids, or 12-18 amino acids. In some embodiments, a target peptide for presentation by an MHC class I molecule can be from 7 to 15 amino acids, or 8-12 amino acids.

In accordance with the methods disclosed herein, a target peptide can be associated with the two MHC polypeptide chains in the trimer through non-covalent interactions; or a target peptide can be attached covalently to at least one of the two MHC polypeptide chains in the trimer. As described herein, a covalent linkage between a peptide and an MHC polypeptide chain in the trimer can be achieved through a peptide linker, and alternatively or additionally, through a disulfide bond.

In some embodiments where the MHC molecule is a class I molecule, a target peptide, an MHC class I α chain or a portion thereof, a β2 microglobulin or a portion thereof, can be associated with each other through non-covalent interactions to form a peptide-MHC trimer wherein the target peptide is bound in a peptide-binding grove formed by the α chain or the portion thereof and the β2 microglobulin molecule or the portion thereof. In some embodiments where the MHC molecule is a class I molecule, a target peptide can be covalently linked to either an MHC class I α chain or a portion thereof, or a β2 microglobulin chain or a portion thereof, to form a peptide-MHC trimer wherein the target peptide is bound in a peptide-binding grove formed by the α chain or the portion thereof and the β2 microglobulin or the portion thereof. In some embodiments where the MHC molecule is a class I molecule, a target peptide, an MHC class I α chain or a portion thereof, a β2 microglobulin or a portion thereof, can be linked with each other through peptide linkers to form a single chain peptide-MHC trimer wherein the target peptide is bound in a peptide-binding grove formed by the α chain or the portion thereof and the β2 microglobulin molecule or the portion thereof. Examples of single chain target peptide-MHC trimers include, from N to C terminus, target peptide-β2 microglobulin-MHC class I α chain or a portion thereof, wherein “-” represents a peptide linker. Single chain target peptide-MHC trimers can include disulfide bonds formed between a cysteine residue in a target peptide or a peptide linker and a cysteine residue in the MHC class I α chain or a portion thereof, as described herein.

In some embodiments where the MHC molecule is a class II molecule, a target peptide, an MHC class II α chain or a portion thereof, and an MHC class II β chain or a portion thereof, can be associated with each other through non-covalent interactions to form a peptide-MHC trimer wherein the target peptide is bound in a peptide-binding grove formed by the α chain or the portion thereof and the β chain or the portion thereof. Alternatively, a target peptide can be covalently linked to an MHC class II α chain or a portion thereof or to an MHC class II β chain or a portion thereof, e.g., through a peptide linker, optionally also through a disulfide bond, wherein the target peptide is bound in a peptide-binding grove formed by the α chain or the portion thereof and the β chain or the portion thereof. Irrespective of whether a target peptide is not covalently linked to either MHC class II polypeptide chain or is covalently linked to at least one MHC class II polypeptide chain, the two MHC II polypeptide chains can be linked to peptide sequences of a binding pair, respectively. Binding pairs are described herein and include, for example, a Jun-Fos zipper and knobs-into-holes. For instance, a Jun-Fos zipper can comprise a Jun leucine zipper dimerization motif and a Fos leucine zipper dimerization motif. In some embodiments, one of the two MHC II polypeptide chains in the trimer is attached (i.e., covalently linked), e.g., through a peptide linker, to a Jun leucine zipper dimerization motif, and the other MHC II polypeptide chain is covalently linked, e.g., through a peptide linker, to a Fos leucine zipper dimerization motif. In some embodiments, a Jun leucine zipper dimerization motif and a Fos leucine zipper dimerization motif are attached to the C-terminus of the two MHC II polypeptide chains, respectively. In some embodiments, a peptide-MHC class II trimer comprises, (i) from N- to C-terminus, a target peptide-α chain or a portion thereof of an MHC class II molecule-one of a Jun leucine zipper dimerization motif and a fos leucine zipper dimerization motif, and (ii) from N- to C-terminus: β chain or a portion thereof of the MHC class II molecule-the other one of a Jun leucine zipper dimerization motif and a fos leucine zipper dimerization motif (wherein the hyphen “-” represents a peptide linker), such as those described in WO2021/113297A1 (Regeneron Pharmaceuticals, Inc.), incorporated herein by reference. In some embodiments, a peptide-MHC class II trimer comprises, (i) from N- to C-terminus, a target peptide-β chain or a portion thereof of an MHC class II molecule-one of a Jun leucine zipper dimerization motif and a fos leucine zipper dimerization motif, and (ii) from N- to C-terminus: α chain or a portion thereof of the MHC class II molecule-the other one of a Jun leucine zipper dimerization motif and a fos leucine zipper dimerization motif (wherein the hyphen “-” represents a peptide linker), such as those described in WO 2021/113297A1 (Regeneron Pharmaceuticals, Inc.), incorporated herein by reference).

### Control Peptide and Blocking Reagent

The methods disclosed herein utilizes a blocking reagent that comprises a peptide-MHC trimer wherein the peptide in the MHC trimer is a control peptide.

A control peptide as used herein includes peptides that differ from a target peptide to enable identification and isolation of cells expressing antibody molecules specific for an interface between the target peptide and an MHC molecule. A control peptide can have the same or different length as a target peptide, and differs from a target peptide by one or more amino acids. If differences occur at multiple amino acid positions, the positions may be consecutive or not consecutive. In some embodiments, a control peptide differs from a target peptide by 1 amino acid. In some embodiments, a control peptide differs from a target peptide by 2 or more amino acids. In some embodiments, a control peptide differs from a target peptide by 3 or more amino acids. In some embodiments, a control peptide differs from a target peptide by 4 or more amino acids. In some embodiments, a control peptide differs from a target peptide by 5 or more amino acids. In some embodiments, a control peptide differs from a target peptide by 6 or more amino acids. In some embodiments, a control peptide differs from a target peptide in at least 10% of the amino acids. In some embodiments, a control peptide differs from a target peptide in at least 20% of the amino acids. In some embodiments, a control peptide differs from a target peptide in at least 30% of the amino acids. In some embodiments, a control peptide differs from a target peptide in at least 40% of the amino acids. In some embodiments, a control peptide differs from a target peptide in at least 50% of the amino acids. In some embodiments, a control peptide differs from a target peptide in at least 60% of the amino acids. In some embodiments, a control peptide differs from a target peptide in at least 70% of the amino acids. In some embodiments, a control peptide differs from a target peptide in at least 80% of the amino acids. In some embodiments, a control peptide differs from a target peptide in at least 90% of the amino acids. In some embodiments, a control peptide differs from a target peptide in 100% of the amino acids, i.e., the peptides do not share any common amino acids. In embodiments where a control peptide differs significantly from a target peptide, e.g., differing in at least 50% of the amino acids, such a control peptide can be considered an “irrelevant peptide”.

In some embodiments, a control peptide is an off-target peptide. The term “off-target peptide” as used herein refers to a peptide similar to a target peptide presented by an MHC molecule, such that an antigen-recognition molecule (e.g., a TCR) for the intended target peptide-MHC trimer is likely to also recognize an off-target peptide-MHC trimer. One challenge in developing drugs targeting pHLA complexes is that other proteome-derived peptides can form pHLA complexes resembling the target complex, which can cause normal tissue toxicity resulting from off-target therapeutic delivery. Therefore, using an off-target peptide-MHC trimer in a blocking reagent will permit identification of cells expressing antibody molecules against a target peptide-MHC interface with high specificity. Candidate off-target peptides can be identified by applying an in-silico computational method that predicts peptides with the greatest possibility of forming off-target pHLA complexes similar to the targeted complex using Peptide In Groove Similarity Predictor (PIGSPRED), as described in e.g., WO2023/122621A2 (Regeneron Pharmaceuticals, Inc.), incorporated herein by reference. For example, PIGSPRED uses netMHCpan [PMID: 32406916] machine-learning tool to quantitatively predict peptide binding to MHC class-I molecules. For a peptide-HLA pair, the tool computes IC₅₀ value, percentile rank of predicted binding affinity (BA) score compared to set of random peptides (% Rank_BA), and percentile rank of predicted eluted ligand (EL) score compared to set of random peptides (% Rank_EL) to evaluate binding. A Degree of Similarity (DoS) score can be computed for each off-target peptide to quantify the homology between a potential off-target and the target peptide. The DoS score can be based in part on the number of amino acids in an off-target peptide identical to amino acids at corresponding positions of the target peptide that are considered to be important for binding to an antigen-recognition molecule. Once the DoS score is determined, the number of off-targets at different threshold values can be computed to compare the likelihood of off-target toxicity. In some embodiments, a control peptide is an off-target peptide having a DoS of 6 or higher. In some embodiments, a control peptide is an off-target peptide having a DoS of 7 or higher. In some embodiments, a control peptide is an off-target peptide having a DoS of 8 or higher. In some embodiments, a control peptide is an off-target peptide having a DoS of 9 or higher.

In accordance with the methods disclosed herein, a control peptide can be associated with the two MHC polypeptide chains in the trimer through non-covalent interactions; or a control peptide can be covalently attached to at least one of the two MHC polypeptide chains in the trimer. As described herein, a covalent linkage between a peptide and an MHC polypeptide chain in the trimer can be achieved through a peptide linker, and alternatively or additionally, through a disulfide bond.

In some embodiments where the MHC molecule is a class I molecule, a control peptide, an MHC class I α chain or a portion thereof, a β2 microglobulin or a portion thereof, can be associated with each other through non-covalent interactions to form a peptide-MHC trimer wherein the control peptide is bound in a peptide-binding grove formed by the α chain or the portion thereof and the β2 microglobulin molecule or the portion thereof. In some embodiments where the MHC molecule is a class I molecule, a control peptide can be covalently linked to either an MHC class I α chain or a portion thereof, or a β2 microglobulin chain or a portion thereof, to form a peptide-MHC trimer wherein the control peptide is bound in a peptide-binding grove formed by the α chain or the portion thereof and the β2 microglobulin molecule or the portion thereof. In some embodiments where the MHC molecule is a class I molecule, a control peptide, an MHC class I α chain or a portion thereof, a β2 microglobulin or a portion thereof, can be linked with each other through peptide linkers to form a single chain peptide-MHC trimer wherein the control peptide is bound in a peptide-binding grove formed by the α chain or the portion thereof and the β2 microglobulin molecule or the portion thereof. Examples of single chain control peptide-MHC trimers include, from N to C terminus, control peptide-β2 microglobulin-MHC class I α chain or a portion thereof, wherein “-” represents a peptide linker. Single chain control peptide-MHC trimers can include disulfide bonds formed between a cysteine residue in a control peptide or a peptide linker and a cysteine residue in the MHC class I α chain or a portion thereof, as described herein.

In some embodiments where the MHC molecule is a class II molecule, a control peptide, an MHC class II α chain or a portion thereof, and an MHC class II β chain or a portion thereof, can be associated with each other through non-covalent interactions to form a peptide-MHC trimer wherein the control peptide is bound in a peptide-binding grove formed by the α chain or the portion thereof and the β chain or the portion thereof. In some embodiments where the MHC molecule is a class II molecule, a control peptide can be covalently linked to an MHC class II α chain or a portion thereof or to an MHC class II β chain or a portion thereof, e.g., through a peptide linker, optionally also through a disulfide bond, wherein the control peptide is bound in a peptide-binding grove formed by the α chain or the portion thereof and the β chain or the portion thereof. Irrespective of whether a control peptide is not covalently linked to either MHC class II polypeptide chain or is covalently linked to at least one MHC class II polypeptide chain, the two MHC II polypeptide chains can be linked to peptide sequences of a binding pair, respectively. Binding pairs are described herein and include, for example, a Jun-Fos zipper and knobs-into-holes. For instance, a Jun-Fos zipper can comprise a Jun leucine zipper dimerization motif and a Fos leucine zipper dimerization motif. In some embodiments, one of the two MHC II polypeptide chains in the trimer is attached (i.e., covalently linked), e.g., through a peptide linker, to a Jun leucine zipper dimerization motif, and the other MHC II polypeptide chain is covalently linked, e.g., through a peptide linker, to a Fos leucine zipper dimerization motif. In some embodiments, a Jun leucine zipper dimerization motif and a Fos leucine zipper dimerization motif are attached to the C-terminus of the two MHC II polypeptide chains, respectively. In some embodiments, a peptide-MHC class II trimer comprises, (i) from N- to C-terminus, a control peptide-α chain or a portion thereof of an MHC class II molecule-one of a Jun leucine zipper dimerization motif and a fos leucine zipper dimerization motif, and (ii) from N- to C-terminus: β chain or a portion thereof of the MHC class II molecule-the other one of a Jun leucine zipper dimerization motif and a fos leucine zipper dimerization motif (wherein the hyphen “-” represents a peptide linker), such as those described in WO2021113297 (Regeneron Pharmaceuticals, Inc.), incorporated herein by reference. In some embodiments, a peptide-MHC class II trimer comprises, (i) from N- to C-terminus, a control peptide-β chain or a portion thereof of an MHC class II molecule-one of a Jun leucine zipper dimerization motif and a fos leucine zipper dimerization motif, and (ii) from N- to C-terminus: α chain or a portion thereof of the MHC class II molecule-the other one of a Jun leucine zipper dimerization motif and a fos leucine zipper dimerization motif (wherein the hyphen “-” represents a peptide linker), such as those described in WO2021113297 (Regeneron Pharmaceuticals, Inc.), incorporated herein by reference.

Blocking Reagent v. Sorting Reagent

The methods disclosed herein utilize a blocking reagent comprising a peptide-MHC trimer comprising a control peptide and two MHC polypeptide chains, wherein the control peptide is presented in (bound to) a peptide-binding groove formed by the two MHC polypeptide chains. In some embodiments, the blocking reagent comprises 2 or more peptide-MHC trimers having different control peptides. In some embodiments, the blocking reagent comprises 2 peptide-MHC trimers having 2 different control peptides. In some embodiments, the blocking reagent comprises 3 peptide-MHC trimers having 3 different control peptides. In some embodiments, the blocking reagent comprises 4 peptide-MHC trimers having 4 different control peptides. In some of the embodiments where a blocking reagent comprises 2 or more peptide-MHC trimers having different control peptide, the two MHC polypeptide chains may be identical among the 2 or more peptide-MHC trimers; or alternatively, may not be identical among the 2 or more peptide-MHC trimers provided that the peptide-binding grooves formed by the two MHC polypeptide chains in each trimer are substantially identical. For example, one control peptide-MHC trimer may be a control peptide-MHC I trimer which includes a portion of an MHC I α chain comprising α1, α2 and α3 domains, but not the full ectodomain of the α chain, while another control peptide-MHC I trimer may include the full ectodomain of the same MHC I α chain. As another example, one control peptide-MHC trimer may be a control peptide-MHC II trimer which includes a portion of an MHC II α chain comprising α1 and α2 domains, but not the full ectodomain of the α chain, while another control peptide-MHC II trimer may include the full ectodomain of the same MHC II α chain. As still another example, one control peptide-MHC trimer may include a disulfide bond between a peptide linker connecting the control peptide to one of the two MHC polypeptide chains, while another control peptide-MHC trimer does not include a disulfide bond.

Further, the methods disclosed herein utilize a sorting reagent comprising a peptide-MHC trimer which comprises a target peptide and two MHC polypeptide chains, wherein the target peptide is presented in (bound to) a peptide-binding groove formed by the two MHC polypeptide chains. The two MHC polypeptide chains may be identical with the two polypeptide chains in a peptide-MHC trimer in the blocking reagent; or alternatively, may not be identical with the two polypeptide chains in a peptide-MHC trimer in the blocking reagent provided that the peptide-binding groove formed by the two MHC polypeptide chains in the trimer of the sorting reagent is substantially identical to the peptide-binding groove(s) formed by the two MHC polypeptide chains in the trimer(s) of the blocking reagent to achieve effective blocking. For example, one target peptide-MHC trimer may be a target peptide-MHC trimer which includes a portion of an MHC I α chain comprising α1, α2 and α3 domains, but not the full ectodomain of the α chain, while a control peptide-MHC I trimer may include the full ectodomain of the same MHC I α chain. As another example, a control peptide-MHC trimer may include a disulfide bond between a peptide linker connecting the control peptide to one of the two MHC polypeptide chains, while a target peptide-MHC trimer does not include a disulfide bond. It is beneficial to have the two MHC polypeptide chains in the trimer(s) of a blocking reagent to encompass and cover non-PiG specific epitopes that are included in an immunogen used to immunize an animal and also shared by the peptide-MHC trimer in the sorting reagent.

In accordance with the present methods, the target peptide-MHC trimer in a sorting reagent is labeled to permit identification of cells expressing antibody molecules specific to a target peptide-MHC interface. Suitable labels and labeling approaches are further described hereinbelow. The control peptide-MHC trimers in a blocking reagent may be unlabeled, or alternatively, labeled in a manner that differentiates from the label of the target peptide-MHC trimer in the sorting reagent. In some embodiments, the control peptide-MHC trimer(s) in a blocking reagent is(are) labeled. In some embodiments, the target peptide-MHC trimer in a sorting reagent is labeled with a first fluorescent compound, and the control peptide-MHC trimer(s) in a blocking reagent is(are) labeled with a second fluorescent compound that differentiates from the first fluorescent compound to permit differentiation and sorting in 2-color FACS.

Depending on whether a blocking reagent is labeled or not, the concentration of the peptide-MHC trimers in the blocking reagent relative to the concentration of the sorting reagent will vary. In embodiments where the blocking reagent is labeled (labeled differently from the sorting reagent), the total molar concentration of the control peptide-MHC trimer(s) in the blocking reagent is comparable to or higher than the molar concentration of the target peptide-MHC trimer in the sorting reagent. By “comparable” it is meant that the molar concentrations of the control peptide-MHC trimer(s) in the blocking reagent and the molar concentration of the target peptide-MHC trimer in the sorting reagent are similar, e.g., within 30%, 25%, 20%, or 10% of variation or margin of each other. In embodiments wherein the total molar concentration of the control peptide-MHC trimer(s) in the blocking reagent is higher than the molar concentration of the target peptide-MHC trimer in the sorting reagent, it can be several fold higher (e.g., 2 fold, 3 fold, 4 fold, 5 fold, 6 fold, and up to 10 fold), or more than several fold higher such as 100 fold higher. In embodiments where the blocking reagent is unlabeled, the total molar concentration of the control peptide-MHC trimer(s) in the blocking reagent is in excess, e.g., at least 10 fold, relative to the molar concentration of the target peptide-MHC trimer in the sorting reagent. In some embodiments, the total molar concentration of the control peptide-MHC trimer(s) in the blocking reagent is at least 10-100 fold relative to the molar concentration of the target peptide-MHC trimer in the sorting reagent. In some embodiments, the total molar concentration of the control peptide-MHC trimer(s) in the blocking reagent is at least 20 fold relative to the molar concentration of the target peptide-MHC trimer in the sorting reagent. In some embodiments, the total molar concentration of the control peptide-MHC trimer(s) in the blocking reagent is at least 30 fold relative to the molar concentration of the target peptide-MHC trimer in the sorting reagent. In some embodiments, the total molar concentration of the control peptide-MHC trimer(s) in the blocking reagent is at least 40 fold relative to the molar concentration of the target peptide-MHC trimer in the sorting reagent. In some embodiments, the total molar concentration of the control peptide-MHC trimer(s) in the blocking reagent is at least 50 fold relative to the molar concentration of the target peptide-MHC trimer in the sorting reagent. In some embodiments, the total molar concentration of the control peptide-MHC trimer(s) in the blocking reagent is at least 75 fold relative to the molar concentration of the target peptide-MHC trimer in the sorting reagent. In some embodiments, the total molar concentration of the control peptide-MHC trimer(s) in the blocking reagent is at least 100 fold relative to the molar concentration of the target peptide-MHC trimer in the sorting reagent. In some embodiments, the total molar concentration of the control peptide-MHC trimer(s) in the blocking reagent is at least 150 fold relative to the molar concentration of the target peptide-MHC trimer in the sorting reagent. In some embodiments, the total molar concentration of the control peptide-MHC trimer(s) in the blocking reagent is at least 200 fold relative to the molar concentration of the target peptide-MHC trimer in the sorting reagent. In embodiments where multiple unlabeled control peptide-MHC trimers are included in a blocking reagent, each control peptide-MHC trimer can be used at a molar concentration in excess, e.g., at least 10-100 fold, relative to molar concentration of the target peptide-MHC trimer in the sorting reagent.

In accordance with the methods disclosed herein, the peptide-MHC trimer in the blocking reagent and/or the sorting reagent can also be provided in a multimeric form, for example, a dimer, trimer, tetramer, pentamer, hexamer of the peptide-MHC trimer, or a mixture thereof. By a multimeric form of a peptide-MHC trimer, it is meant that multimer units of the peptide-MHC trimer are present. In some embodiments, the peptide-MHC trimer in the blocking reagent and/or the sorting reagent is provided in both monomeric and multimeric forms. In embodiments where a multimeric form of a peptide-MHC trimer is employed, the multimeric form can be formed by utilizing a multivalent molecule to which the peptide-MHC trimer is bound or linked. A multivalent molecule can be a dimer, trimer, tetramer, pentamer, hexamer, and the like, or a combination thereof. In some embodiments, the multivalent molecule is a streptavidin multimer (e.g., tetramer), which can be complexed with biotin which is then linked to a peptide-MHC trimer, thereby providing a multimeric (e.g., tetrameric) form of the peptide-MHC trimer. In some embodiments, a streptavidin multimer includes tetramer and may additionally include trimer and/or dimer. In some embodiments, a streptavidin multimer is conjugated with a fluorophore such as phycoerythrin. In some embodiments, the multivalent molecule is a dimer of an immunoglobulin Fc fragment, to which a peptide-MHC trimer can be linked to provide a dimeric form of the peptide-MHC trimer. In some embodiments, the multivalent molecule is a trimer of a trimerization molecule, such as foldon, to which a peptide-MHC trimer can be linked to provide a trimeric form of the peptide-MHC trimer.

### Method Steps

To practice the present methods, a population of antibody-producing cells encompassing cells that express on the cell surface antibody molecules specific for an interface between a target peptide and an MHC molecule is contacted with a blocking reagent. The cells can be incubated with the block reagent for a period of 10 to 15 minutes.

After the cells have been incubated with the blocking reagent, and without removing the blocking reagent, the cell population is contacted with a sorting reagent in the presence of the blocking reagent. The cells can be incubated with the sorting reagent in the presence of the blocking reagent for a period of 30 to 60 minutes.

Thereafter, the cells are washed to remove unbound sorting reagent and unbound blocking reagent. Appropriate wash buffers comprise phosphate buffered saline.

After the wash step, cells that remain bound to the sorting reagent can be collected to obtain cells expressing antibody molecules specific for an interface between the target peptide and the MHC molecule.

### Antibody-Producing Cells

The terms “antibody-producing cells” and “antibody-expressing cells” refer to cells that express antibody molecules on the cell surface, i.e., the antibody molecules are bound to or anchored in the cell membrane. Cell surface expression of antibody molecules can occur either naturally, e.g., as a result of B-cell activation or as a result of recombinant technology and genetic engineering. The term, therefore, encompasses lymphocytes of antigen-dependent B-cell lineage, including memory B-cells, as well as recombinant cells such as non-lymphoid cells engineered to express antibody molecules on the cell surface, including yeast and mammalian cells such as immortalized cells and hybridoma cells. In some embodiments, the immortalized cells include Chinese hamster ovary (CHO) cells, human embryonic kidney (HEK)293 cells, and murine myeloma cells (e.g., NS0 and Sp2/0).

In some embodiments, antibody-producing cells are primary antibody-producing cells. “Primary cells” refers to cells grown outside of their natural environment, such as tissue cells isolated from a mammal. In certain embodiments, primary antibody-producing cells are tissue-derived, such as from spleen, lymph node, bone marrow, or peripheral blood. In some embodiments, antibody-producing cells may be derived from primary antibody-producing cells. For example, primary antibody-producing cells may be fused to myeloma cells to make hybridomas, or otherwise immortalized, such as infected with a virus (e.g., EBV), or may be differentiated by cell sorting techniques based on protein markers expressed by particular B cell types.

In some embodiments, antibody-producing cells are mammalian cells or yeast engineered to express antibody molecules on the cell surface. In the context of cells engineered to express antibody molecules, the cells may be engineered to express full length immunoglobulin molecules or antigen-binding fragments. In some embodiments, antibody-producing cells are yeast cells (e.g., S. cerevisiae or Pichia) engineered to express antibody molecules on the cell surface. In some embodiments, antibody-producing cells are mammalian cells engineered to express antibody molecules on the cell surface. In some embodiments, the mammalian cells are immortalized cells engineered to express antibody molecules on the cell surface, which include, e.g., CHO cells, HEK293 cells, and murine myeloma cells (e.g., NS0 and Sp2/0).

Some methodologies described herein also apply to cell surface display platforms of various host cells, including yeast or mammalian cells such as CHO cells, that express antibody molecules on the cells surface to permit screening from antibody gene libraries or repertoires of antibody maturation variants.

Yeast surface display (YSD) platforms have been described and widely used in antibody screening (Border and Wittrup, Nat Biotechnol. 1997; 15:553-7; Feldhaus M J et al., Nat Biotechnol. 2003; 21:163-70; McMahon C et al., Nat Struct Mol Biol. 2018; 25:289-96). Display of Fab regions on the yeast surface has been reported to increase the antibody diversity and enlarge the library size (Weaver-Feldhaus J M et al., FEBS Lett. 2004; 564: 24-34; Rosowski S et al., Microb Cell Fact. 2018; 17:3; Sivelle C et al., MAbs. 2018; 10:720-9).

Mammalian cell surface display platforms that display full-length antibodies or Fab fragments on the surface of mammalian cells, including CHO cells, have been described, e.g., by Zhou et al. MAbs. 2010; 2(5): 508-518; Nguyen et al., Protein Engineering, Design & Selection, 2018, vol. 31 no. 3, pp. 91-101). In addition, suitable for use herein are mammalian cells that carry a single antibody gene can be transfected with a gene encoding activation induced deaminase (AID) which initiates somatic hypermutation (SHM) by converting deoxycytidines (dC) to deoxyuracils (dU), to mutate the antibody gene in cells during cell proliferation in the cell culture. See, e.g., Chen C. et al., Biotechnol Bioeng. 113, 39-51 (2016).

### Immunization and Collection of Primary Antibody-Producing Cells

Immunization of mammals including human and nonhuman animals can be done by any methods known in the art (see, for example, E. Harlow and D. Lane—Antibodies A Laboratory Manual, Cold Spring Harbor (1988); Malik and Lillehoj, Antibody techniques: Academic Press, 1994, CA). A target peptide MHC trimer can be administered as an protein immunogen, or alternatively, a DNA plasmid can be administered that expresses the peptide components and produces the trimer (including embodiments where the trimer is a single chain trimer) in vivo. In some embodiments, the two MHC polypeptide chains in the target peptide-MHC trimer used for immunization are identical to the two MHC polypeptide chains in the target peptide-MHC trimer used in the sorting reagent. In other embodiments, the two MHC polypeptide chains in the target peptide-MHC trimer used for immunization are not identical to the two MHC polypeptide chains in the target peptide-MHC trimer used in the sorting reagent, yet have sufficient identity to form the same peptide-binding groove to present the target peptide. For instances, the MHC polypeptide chains may differ in that one polypeptide chain may include a cysteine substitution that permits a disulfide bond formation with the peptide to be presented or a peptide linker connecting the peptide to be presented to the MHC polypeptide chain. In some embodiments, the two MHC polypeptide chains in the target peptide-MHC trimer used for immunization, the two MHC polypeptide chains in the target peptide-MHC trimer used in the sorting reagent, and the two MHC polypeptide chains in a control-peptide-MHC trimer used in the blocking reagent are all identical or have sufficient sequence identity to form the same peptide-binding groove.

A target peptide MHC trimer may be administered directly to a mammal, without adjuvant, or with adjuvant to aid in stimulation of the immune response. Adjuvants known in the art include, but are not limited to, complete and incomplete Freund's adjuvant, MPL+TDM adjuvant system (Sigma), or RIBI (muramyl dipeptides) (see O'Hagan, Vaccine Adjuvant, by Human Press, 2000, NJ).

Once an appropriate immune response has been achieved, antibody-producing cells are collected from the immunized animal.

Antibody-producing cells can be collected from different sources of an immunized animal, including but not limited to spleen, lymph node, bone marrow, and peripheral blood. In some embodiments, following immunization, splenocytes are harvested from an immunized animal. In some embodiments, peripheral blood mononuclear cells (PBMCs) are harvested from an immunized animal.

In some embodiments of the methods, a population of antibody-producing cells are antibody-producing B cells. In some embodiments, antibody-producing B cells can be obtained from immunized animals and isolated by FACS based on cell-surface B cell markers. B cell markers are known in the art. For example, applicable B cell markers that can be detected through the use of FACS include, but are not limited to, IgG, IgM, IgE, IgA, IgD, CD1, CD5, CD19, CD20, CD21, CD22, CD23, CD24, CD25, CD27, CD30, CD38, CD40, CD78, CD80, CD138, CD319, TLR4, IL-6, PDL-2, CXCR3, CXCR4, CXCR5, CXCR6, IL-10, and TGFβ.

In some embodiments, following immunization, splenocytes are harvested from an immunized animal. Following removal of red blood cells by lysis, IgG-positive antigen-positive B cells can be isolated and used as an antibody producing cell population in the method.

In some embodiments, peripheral blood mononuclear cells (PBMCs) are harvested from an immunized animal known to have humoral immunity to an antigen of interest. IgG-positive, antigen-positive B cells can then be isolated for use as antibody producing cells in the present method.

Collected primary antibody-producing cells, such as antibody-producing B cells, can be processed to enrich for cells that express antibody on the cell surface that is directed to an antigen of interest such as a target peptide-MHC trimer. In some embodiments, an initial step of purification is optionally performed to enrich for primary antibody-producing cells. Such purification includes affinity chromatography, also referred to as affinity purification. There are several types of affinity purification known in the art, such as ammonium sulfate precipitation, affinity purification with immobilized protein A, G, A/G, or L; and affinity purification with immobilized antigen.

### Label

A peptide-MHC trimer can be labeled with small molecules, radioisotopes, enzymatic proteins and fluorescent dyes. In some embodiments, the detectable label is a small molecule. Detectable small molecule labels allow for easy labeling of proteins and can be used in a number of regularly deployed detection assays known in the art.

In some embodiments, the detectable label is an enzyme reporter. Enzyme labels are larger than biotin, however, they rarely disrupt antibody function. Commonly used enzyme labels are horseradish peroxidase (HRP), alkaline phosphatase (AP), glucose oxidase and β-galactosidase. To use enzyme-labeled antibodies, samples are incubated with an enzyme-specific substrate that is catalyzed by the enzyme to produce a colored product (chromogenic assays) or light (chemiluminescent assays). Each enzyme has a set of substrates and detection methods that can be employed. For example, HRP can be reacted with diaminobenzidine to produce a brown-colored product or with luminol to produce light. In contrast, AP can be reacted with para-Nitrophenylphosphate (pNPP) to produce a yellow-colored product detected by a spectrophotometer or with 5-bromo-4-chloro-3-indolyl phosphate (BCIP) and nitroblue tetrazolium (NBT) to produce a purple-colored precipitate.

In some embodiments, the detectable label is a fluorescent label. Fluorescent tags can be covalently attached to a protein through primary amines or thiol.

A label can be attached to a peptide-MHC trimer directly or indirectly. Indirect labeling can utilize binding pairs such as biotin and streptavidin. For instance, a peptide-MHC trimer can be labeled with biotin, which can bind to streptavidin conjugated with a fluorescent compound.

### Fluorescence-Activated Cell Sorting (FACS)

Flow cytometry is a popular analytical cell-biology technique that utilizes light to count and profile cells in a heterogenous fluid mixture. Flow cytometry is a particularly powerful method because it allows a researcher to rapidly, accurately, and simply collect data related to many parameters from a heterogeneous fluid mixture containing live cells. Fluorescence-Activated Cell Sorting (FACS) is a derivative of flow cytometry that adds an exceptional degree of functionality. Using FACS a researcher can physically sort a heterogeneous mixture of cells into different populations.

Two-dimensional (2D) FACS is the sorting of cells based on two different fluorescent labels. Two-dimensional FACS provides more selective results than FACS based on one parameter. Two-dimensional FACS is available to be used in embodiments of this disclosure where a blocking reagent is also labeled in addition to the sorting reagent being labeled. In some embodiments where the first detectable label is a first fluorescent label, and the second detectable label is a second fluorescent label that differentiates from the first fluorescent label, two-dimensional FACS is used to collect cells that remain bound to one of the labels. In specific embodiments, the first detectable label is A647 and the second detectable label is Phycoerythrin.

### Obtaining a Population of Cells Enriched in Cells Expressing Antibody Molecules Specific for a Peptide-MHC Interface

The antibody-producing cells that remain bound to the sorting reagent are collected. This collected cells are enriched in cells expressing antibody molecules binding to a target peptide-MHC interface.

The collected cells can be sorted or separated into single cells. In some embodiments, fluorescence-activated cell sorting (FACS) is used to sort and select single antibody-producing cells. Protocols for single cell isolation by flow cytometry are well-known (Huang, J. et al, 2013, supra). Single antibody-producing cells may be sorted and collected by alternative methods known in the art, including but not limited to manual single cell picking, limited dilution, B cell panning of adsorbed antigen, microfluidics, laser capture microdissection, and Gel Bead Emulsions (GEMs), which are all well-known in the art. See, for example, Rolink et al., J Exp Med (1996) 183:187-194; Lightwood, D. et al, J. Immunol. Methods (2006) 316(1-2):133-43; Gross et al., Int. J. Mol. Sci. (2015) 16: 16897-16919; and Zheng et al., Nature Communications (2017) 8: 14049. Gel Bead Emulsions (GEMs) are also commercially available (e.g., 10× Chromium System from 10× Genomics, Pleasanton, CA).

Once collected, single antibody-producing cells may be propagated by common cell culture techniques for subsequent DNA preparation. Alternatively, antibody genes may be amplified from single antibody-producing cells directly and subsequently cloned into DNA vectors.

Generating Antibody Molecules from Nucleic Acids Obtained from Antibody-Producing Cells

Nucleic acids encoding an antibody or a fragment thereof can be isolated from the antibody-producing cells obtained using the methods described herein.

In some embodiments, genes or nucleic acids encoding immunoglobulin variable heavy and variable light chains (i.e., VH and VL, and VL can be Vκ or Vλ chain) can be recovered using RT-PCR protocols with nucleic acids isolated from antibody-producing cells. These RT-PCR protocols are well known and conventional techniques, as described for example, by Wang et al., J. Immunol. Methods (2000) 244:217-225 and described herein.

In some embodiments, the nucleic acid encodes a fragment of an antibody, such as a variable domain, constant domain or combination thereof. In certain embodiments, the nucleic acid isolated from an antibody-producing cell encodes a variable domain of an antibody. In some embodiments, the nucleic acid encodes an antibody heavy chain or a fragment thereof (e.g., the variable domain of the antibody heavy chain). In other embodiments, the nucleic acid encodes an antibody light chain or a fragment thereof (e.g., the variable domain of the antibody light chain).

Once recovered, antibody-encoding genes or nucleic acids can be cloned into IgG heavy- and light-chain expression vectors and expressed via transfection of host cells. For example, antibody-encoding genes or nucleic acids can be inserted into a replicable vector for further cloning (amplification of the DNA) or for expression (stably or transiently) in cells. Many vectors, particularly expression vectors, are available or can be engineered to comprise appropriate regulatory elements required to modulate expression of an antibody encoding gene or nucleic acid.

An expression vector in the context of the present disclosure can be any suitable vector, including chromosomal, non-chromosomal, and synthetic nucleic acid vectors (a nucleic acid sequence comprising a suitable set of expression control elements) as described herein. Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In some embodiments, a nucleic acid molecule is included in a naked DNA or RNA vector, including, for example, a linear expression element (as described in, for instance, Sykes and Johnston, Nat Biotech (1997) 12:355-59), a compacted nucleic acid vector (as described in for instance U.S. Pat. No. 6,077,835), or a plasmid vector such as pBR322 or pUC 19/18. Such nucleic acid vectors and the usage thereof are well known in the art. See, for example, U.S. Pat. Nos. 5,589,466 and 5,973,972. In certain embodiments, the expression vector can be a vector suitable for expression in a yeast system. Any vector suitable for expression in a yeast system may be employed. Suitable vectors include, for example, vectors comprising constitutive or inducible promoters such as yeast alpha factor, alcohol oxidase and PGH. See, F. Ausubel et al., ed. Current Protocols in Molecular Biology, Greene Publishing and Wiley InterScience New York (1987); and Grant et al., Methods in Enzymol 153, 516-544 (1987).

In certain embodiments, the vector comprises a nucleic acid molecule (or gene) encoding a heavy chain of the antibody and a nucleic acid molecule (or gene) encoding a light chain of the antibody, wherein the antibody is produced by an antibody-producing cell that has been obtained by a method of the present disclosure.

Host cells suitable for expression of antibody molecules include, but are not limited to, cells of either prokaryotic or eukaryotic (generally mammalian) origin. In some embodiments, the host cell is a bacterial or yeast cell. In some embodiments, the host cell is a mammalian cell. In other embodiments, the host cell can be, for example, a Chinese hamster ovarian cells (CHO) such as, CHO K1, DXB-11 CHO, Veggie-CHO cells; a COS (e.g., COS-7); a stem cell; retinal cells; a Vero cell; a CV1 cell; a kidney cell such as, for example, a HEK293, a 293 EBNA, an MSR 293, an MDCK, aHaK, a BHK21 cell; a HeLa cell; a HepG2 cell; WI38; MRC 5; Colo25; HB 8065; HL-60; a Jurkat or Daudi cell; an A431 (epidermal) cell; a CV-1, U937, 3T3 or L-cell; a C127 cell, SP2/0, NS-0 or MMT cell, a tumor cell, and a cell line derived from any of the aforementioned cells. In a particular embodiment, the host cell is a CHO cell. In a specific embodiment, the host cell is a CHO K1 cell.

It will be appreciated that the full-length antibody nucleic acid sequence or gene may be subsequently cloned into an appropriate vector or vectors. Alternatively, the Fab region of an isolated antibody may be cloned into a vector or vectors in line with constant regions of any isotype. Therefore, any constant region may be utilized in the construction of isolated antibodies, including IgG1, IgG2, IgG3, IgG4, IgM, IgA, IgD, and IgE heavy chain constant regions, or chimeric heavy chain constant regions. Such constant regions can be obtained from any human or animal species depending on the intended use of the antibodies. Also, antibody variable regions or Fab region may be cloned in an appropriate vector(s) for the expression of the protein in other formats, such as ScFv, diabody, etc.

In some embodiments, host cells comprising one or more of antibody-encoding nucleic acids are cultured under conditions that express a full-length antibody, and the antibody can then be produced and isolated for further use. In certain embodiments, the host cell comprises a nucleic acid that encodes a variable domain of an antibody, and the cell is cultured under conditions that express the variable domain. In other embodiments, the host cell comprises a nucleic acid that encodes a variable heavy chain (VH) domain of an antibody, and the cell is cultured under conditions that express the VH domain. In another embodiment, the host cell comprises a nucleic acid that encodes a variable light chain (VL) domain of an antibody, and the cell is cultured under conditions that express the VL domain. In specific embodiments, the host cell comprises a nucleic acid that encodes a VH domain of an antibody and nucleic acid that encodes a VL domain of an antibody, and the cell is cultured under conditions that express the VH domain and the VL domain.

### EXAMPLES

### Example 1. PiG Sort Method for Enriching for Cells Expressing Antibodies Specific to HLA-A2/MAGEA3 aa108-116

This Example describes experiments performed to demonstrate the ability of PiG sort method in enriching for cells expressing antibodies specifically against micro-epitopes of MAGEA3 (Melanoma-associated antigen 3 amino acids 108-116 (ALSRKVAEL, SEQ ID NO: 1) Peptide-in-binding-Groove HLA-A2 complex interface.

Mice were immunized with MAGEA3 (108-116)-hß2m-HLA-A2 ecto single chain trimer protein immunogen containing an Myc-Myc-His (MMH) tag. Spleens from the immunized mice were harvested, and splenocytes were obtained and red blood cells were removed by lysis. The cells were then stained with fluorochrome conjugated antibodies specific to B cell surface markers (IgG in this case) to identify B cells.

Antigen expressing B cells were treated using two different methods for comparison: the direct antigen sort method and the PiG sort method.

In the direct antigen sort method used for this Example, antibody-anchored cells were first contacted with biotin labeled MAGEA3 (108-116)-ß2m-HLA-A2 single chain trimer protein as a sorting reagent. Cells were washed to remove any unbound biotin labeled sorting reagent and then contacted with PE labeled streptavidin. Cells expressing antibodies remaining bound to the biotin labeled sorting reagent were identified through PE labeled streptavidin. This direct antigen sort method would identify cells expressing antibodies to various possible immunogenic epitopes of the sorting reagent MAGEA3 (108-116)-ß2m-HLA-A2 trimer protein.

In the PiG antigen sort method used for this Example, cells were first contacted with a blocking reagent containing an unlabeled single chain trimer protein MAGEA3 aa271-279 (FLWGPRALV, SEQ ID NO: 2, as a control peptide)-ß2m-HLA-A2. After ten to fifteen minutes of pre-incubating at 4° C. with the unlabeled trimer blocking reagent, cells were further exposed to a biotin labeled sort reagent MAGEA3 (108-116)-ß2m-HLA-A2 single chain trimer protein in the presence of the unlabeled trimer blocking reagent. The concentration of the blocking reagent used in the preincubation step was one-hundred-fold of the concentration of the biotin labeled sorting reagent. Cells were then washed to remove any unbound biotin labeled sorting reagent and subsequently contacted with PE labeled streptavidin. Cells expressing antibodies specifically to an HLA-A2/MAGEA3 (108-116) peptide interface remaining bound to the biotin labeled sorting reagent were identified through PE labeled streptavidin.

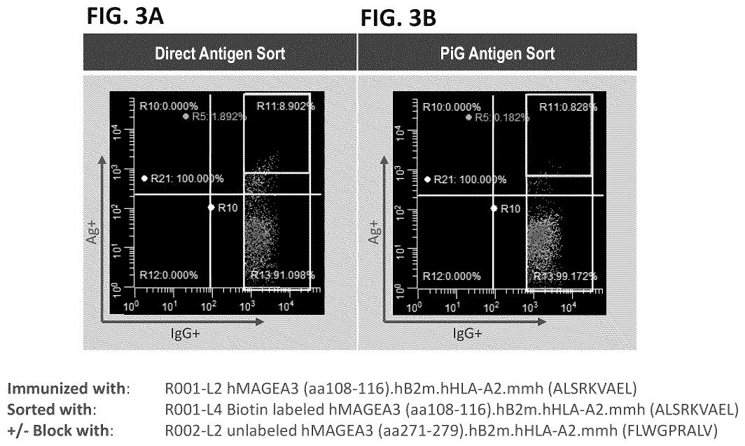

Cells identified by the two methods were then further analyzed using fluorescence activated cell sorting (FACS). As shown in FIG. 3A, 61 B cells out of one million splenocytes expressing antibodies bind to the biotin labeled sort reagent were detected (yellow rectangle) from the mouse using the direct sort method. In contrast, as shown in FIG. 3B, only 9 B cells out of one million splenocytes expressing antibodies specific to the biotin labeled sort reagent were detected (yellow rectangle) using the PiG sort method.

Single B cells that were detected using either the direct antigen sort method or the PiG sort method were sorted and plated. Each individual B cells were subsequently cloned into expression vectors containing human IgG constant regions. Recombinant antibodies were produced from CHO cells after transient transfection. Supernatant from each CHO clone was subsequently collected for Luminex measurement as follows.

Neutravidin coupled beads were incubated for one hour at 25° C. with biotin labeled MAGEA3 (108-116)-ß2m-HLA-A2 single chain trimer protein or biotin labeled MAGEA3 (271-279)-ß2m-HLA-A2 single chain trimer protein to capture the trimer protein onto Neutravidin coupled beads. Neutravidin beads were mixed and washed three times with PBS, and then blocked using 30 mM Biotin (Millipore-Sigma) in 0.15M Tris pH 8.0. Beads were incubated for thirty minutes and washed three times with PBS. Beads were resuspended in PBS 2% BSA 0.05% NaN3 to desired final concentration. For the immunoassays, beads and diluted antibody samples (i.e., supernatants from the CHO antibody clones) were added into each well, and the plates were incubated for two hours at 25° C. and then washed twice with PBS with 0.05% Tween 20. To detect bound antibody levels to individual beads, PE labeled AffiniPure F(ab′)2 Fragment Goat Anti-Mouse IgG, F(ab′)2 Fragment Specific (Jackson Immunoresearch) were added and incubated for thirty minutes at 25° C., followed by two washes. The plates were read in a Luminex FM3D analyzer with xPONENT 4.2 software.

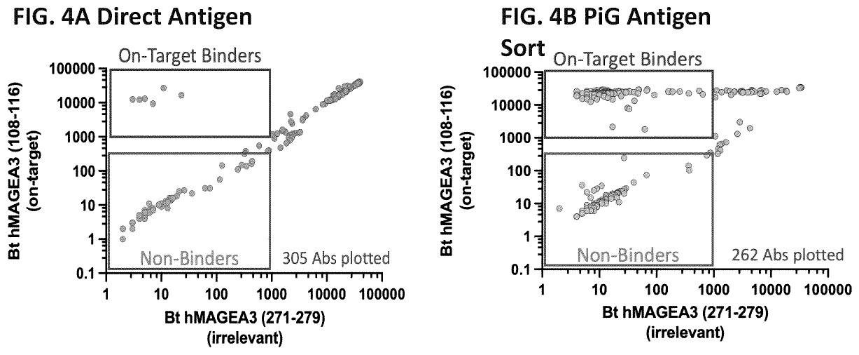

FIG. 4A demonstrates Luminex bindings of clones from the direct antigen sort method. Total three hundred and five clones were tested with biotin labeled MAGEA3 (108-116)-ß2m-HLA-A2 single chain trimer protein, labeled as “on-target” binding signals, and with biotin labeled control peptide MAGEA3 (271-279)-ß2m-HLA-A2 single chain trimer protein, labeled as “irrelevant” binding signals. Here, most of the clones were observed to align diagonally on the graph showing similar binding signals to both the “on-target” and “irrelevant” control peptide HLA-A2/ß2m trimers. This trend indicates that these clones bind common epitopes shared between both the biotin labeled MAGEA3 (108-116)-ß2m-HLA-A2 single chain trimer and the biotin labeled irrelevant peptide MAGEA3 (271-279)-ß2m-HLA-A2 single chain trimer. There was only a very small subset of the clones (n=6 in this case) showing high “on-target” to “irrelevant” binding signal ratios, indicating specific “on-target” binding to the biotin labeled MAGEA3 (108-116)-ß2m-HLA-A2 single chain trimer. These few clones may bind to the epitopes on the biotin labeled MAGEA3 (108-116)-ß2m-HLA-A2 single chain trimer that the biotin labeled irrelevant peptide MAGEA3 (271-279)-ß2m-HLA-A2) single chain trimer does not contain and are called “On-Target Binders”. The unique epitopes of these on-target binders may reside in the specific HLA-A2/MAGEA3 (108-116) interface.

FIG. 4B demonstrates Luminex bindings of clones from the PiG antigen sort method. Total two hundred sixty-two clones were tested. Comparing to FIG. 4A, significantly fewer clones align diagonally on the graph showing similar binding signals to both the “on-target” and “irrelevant” peptide-)-ß2m-HLA-A2 single chain trimers; instead, there was an enrichment of 22 fold of clones falling in the “On-Target Binders” gate showing high “on-target” to “irrelevant” binding ratios. These clones may bind to the unique epitopes on the biotin labeled MAGEA3 (108-116)-ß2m-HLA-A2 single chain trimer that are not shared with the biotin labeled control peptide MAGEA3 (271-279)-ß2m-HLA-A2 single chain trimer. This data showed specific MAGEA3 (108-116) Peptide-in-binding-Groove HLA-A2 complex interface (PiG) selectivity of antibodies isolated from the PiG sort method.

### Example 2: PiG Sort Method for Enriching for Cells Expressing Antibodies Specific to HLA-A2/MAGEA4 Aa230-239

This Example describes experiments performed to demonstrate the ability of PiG sort method in enriching for cells expressing antibodies specifically against micro-epitopes of MAGEA4 (Melanoma-associated antigen 4 amino acids 230-239 (GVYDGREHTV, SEQ ID NO: 3) Peptide-in-binding-Groove HLA-A2 complex interface.

Mice were immunized with MAGEA4 (230-239)-hß2m-HLA-A2 ecto single chain trimer protein immunogen containing T helper cell epitope “PADRE” and Myc-Myc-His (MMH) tag. The PADRE epitope is described by M. F. del Guercio et al., Potent immunogenic short linear peptide constructs composed of B cell epitopes and Pan DR T helper epitopes (PADRE) for antibody responses in vivo. Vaccine 15, 441-448 (1997). Spleens from the immunized mice were harvested, and splenocytes were obtained and red blood cells were removed by lysis. The cells were then stained with fluorochrome conjugated antibodies specific to B cell surface markers (IgG in this case) to identify B cells.

Antigen expressing B cells were treated using two different methods for comparison: the direct antigen sort method and the PiG sort method.

In the direct antigen sort method used for this Example, antibody-anchored cells were first contacted with biotin labeled MAGEA4 (230-239)-ß2m-HLA-A2 single chain trimer protein as a sorting reagent. Cells were washed to remove any unbound biotin labeled sorting reagent and then contacted with PE labeled streptavidin. Cells expressing antibodies remaining bound to the biotin labeled sorting reagent were identified through PE labeled streptavidin. This direct antigen sort method would identify cells expressing antibodies to various possible immunogenic epitopes of the sorting reagent MAGEA4 (230-239)-ß2m-HLA-A2 single chain trimer protein.

In the PiG antigen sort method used for this Example, cells were first contacted with a blocking reagent containing an unlabeled single chain trimer protein hLMP2 (426-434) (CLGGLLTMV, SEQ ID NO: 4, as a control peptide)-ß2m-HLA-A2. After ten to fifteen minutes of pre-incubating with the unlabeled trimer blocking reagent, cells were further exposed to a biotin labeled sort reagent MAGEA4 (230-239)-ß2m-HLA-A2 single chain trimer in the presence of the unlabeled trimer blocking reagent. The concentration of the blocking reagent used in the preincubation step was one-hundred-fold of the concentration of the biotin labeled sorting reagent. Cells were then washed to remove any unbound biotin labeled sorting reagent and subsequently contacted with PE labeled streptavidin. Cells expressing antibodies specifically to an HLA-A2/MAGEA4 (230-239) peptide interface remaining bound to the biotin labeled sorting reagent were identified through PE labeled streptavidin.

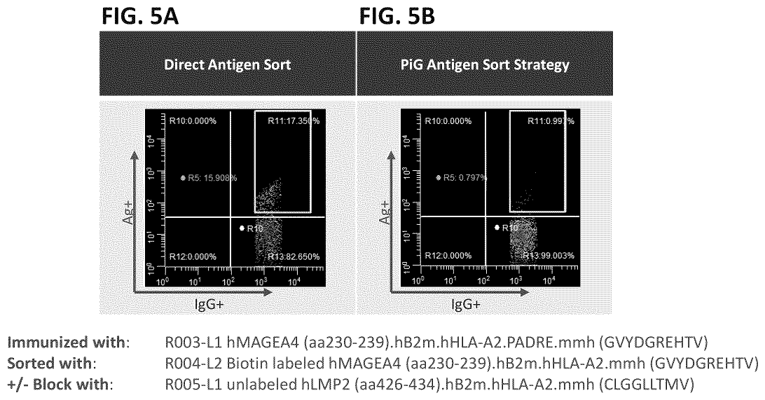

Cells identified by the two methods were then further analyzed using fluorescence activated cell sorting (FACS). As shown in FIG. 5A, 353 B cells out of one million splenocytes expressing antibodies bind to the biotin labeled sort reagent were detected (yellow rectangle) from the mouse using the direct sort method. In contrast, as shown in FIG. 5B, only 28 B cells out of one million splenocytes expressing antibodies specific to the biotin labeled sort reagent were detected (yellow rectangle) using the PiG sort method.

Single B cells that were detected using the PiG sort method were sorted and plated. Each individual B cells were subsequently cloned into expression vectors containing human IgG constant regions. Recombinant antibodies were produced from CHO cells after transient transfection. Supernatant from each CHO clone was subsequently collected for Luminex measurement following the procedure described in Example 1.

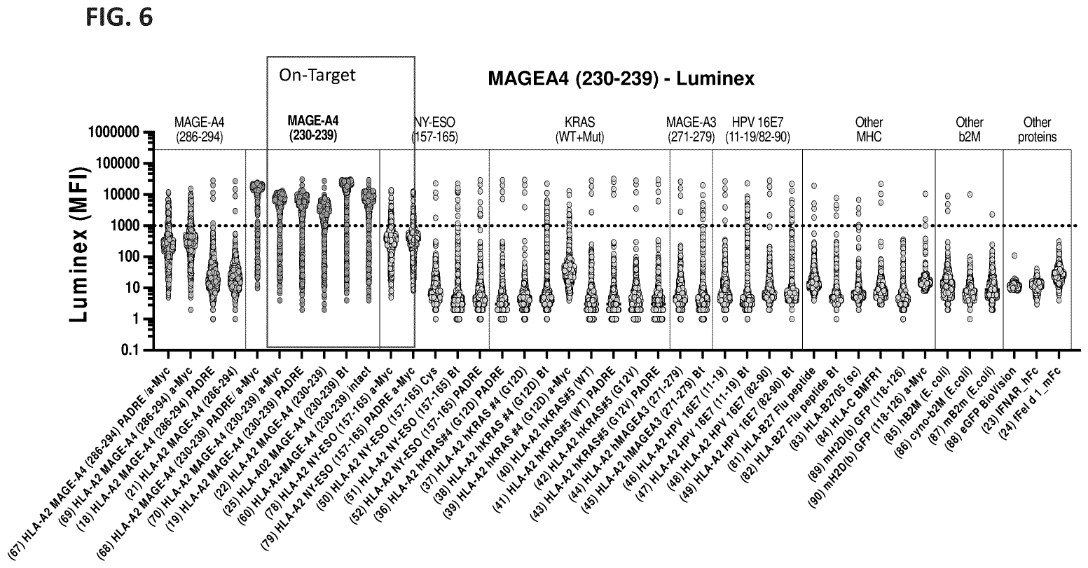

FIG. 6 demonstrates the Luminex binding assay results from the PiG antigen sort method. A series of protein-coupled beads tested in the immunoassay are listed in the X-axis in FIG. 6. Proteins coupled on beads annotated 68, 70, 19, 22, 25, and 60 are the “on-target” HLA-A2/ß2m trimer proteins with MAGEA4 (230-239) peptide, the rest are the HLA-A2/ß2m trimer proteins with “irrelevant” control peptides coupled on beads as annotated 67, 68, 18, 21, 78, 79, 50, 51, 52, 36, 37, 38, 39, 40, 41, 42, 43, 44, 45, 46, 47, 48, 49; or other MHC trimer proteins as annotated 81, 82, 83, 84, 89, 90; or different species beta2-microglobulin (ß2m) proteins as annotated 85, 86, 87; or irrelevant control proteins as annotated 88, 23 and 24. Mean fluorescence intensities (MFI) measured below 1,000 are deemed non-specific bindings as denoted below the dotted line in the graph. Among the total 660 clones tested, the majority of the antibodies showed specific bindings (i.e. above 1,000 MFI) toward the six “on-target” HLA-A2/ß2m trimer proteins as colored in purple in the red rectangle (FIG. 6), while comparably low number of antibodies show specific bindings toward the irrelevant peptide HLA-A2/ß2m trimer proteins (clones either colored in light blue or grey) including MAGEA4 (286-294), NY-ESO(157-165), KRAS (both wild type and mutant peptides), MAGEA3 (271-279), HPV 16E7 (11-19), and HPV 16E7 (82-90), as well as to other MHC trimer proteins HLA-ß27, HLA-C, and mH2D(b), and to human ß2m, monkey ß2m, mouse ß2m proteins, as well as to several control proteins EGFP, IFNAR.hFc, and FelD1.mFc. These results indicate that the majority of the antibodies bind the epitopes residing in the unique “on-target” MAGEA4 (230-239))-ß2m-HLA-A2 interfaces, and only a limited number of antibodies bind the common epitopes shared between the “on-target” HLA-A2/ß2m trimers and the irrelevant HLA-A2/ß2m trimers. The results showed specific MAGEA4 (230-239) Peptide-in-binding-Groove HLA-A2 complex interface (PiG) selectivity of antibodies isolated using the PiG sort method.

### Example 3: PiG Sort Method for Enriching for Cells Expressing Antibodies Specific to HLA-A2/DNTT Aa250-258

This Example describes experiments performed to demonstrate the ability of PiG sort method in enriching for cells expressing antibodies specifically against micro-epitopes of human DNTT (DNA nucleotidylexotransferase) amino acids 250-258 (KLFTSVFGV, SEQ ID NO: 5) Peptide-in-binding-groove HLA-A2 complex interface.

Mice were immunized with human DNTT aa 250-258-hß2m-HLA-A2 ecto single chain trimer protein immunogen containing T helper cell epitope “PADRE” and Myc-Myc-His (MMH) tag. Spleens from the immunized mice were harvested, and splenocytes were obtained and red blood cells were removed by lysis. The cells were then stained with fluorochrome conjugated antibodies specific to B cell surface markers (IgG in this case) to identify B cells.

Antigen expressing B cells were treated using two different methods for comparison: the direct antigen sort method and the PiG sort method.

In the direct antigen sort method used for this Example, antibody-anchored cells were first contacted with biotin labeled DNTT (250-258)-ß2m-HLA-A2 single chain trimer protein as a sorting reagent. Cells were washed to remove any unbound biotin labeled sorting reagent and then contacted with PE labeled streptavidin. Cells expressing antibodies remaining bound to the biotin labeled sorting reagent were identified through PE labeled streptavidin. This direct antigen sort method would identify cells expressing antibodies to various possible immunogenic epitopes of the sorting reagent DNTT (250-258)-ß2m-HLA-A2 single chain trimer protein.

In the PiG antigen sort method used for this Example, cells were first contacted with a blocking reagent containing an unlabeled single chain trimer protein hPOLM (aa238-246)-ß2m-HLA-A2 and an unlabeled single chain trimer protein hPYGL (aa622-630)-ß2m-HLA-A2. hPOLM (aa238-246) (KLFTQIFGV, SEQ ID NO: 6) and hPYGL (aa622-630) (KLITSVADV, SEQ ID NO: 7) are both off target peptides. After ten to fifteen minutes of pre-incubating with the unlabeled trimer blocking reagent, cells were further exposed to a biotin labeled sort reagent DNTT (250-258)-ß2m-HLA-A2 single chain trimer in the presence of the unlabeled blocking reagent. The concentrations of the two control peptide-MHC trimers in the blocking reagent used in the preincubation step were each at one-hundred-fold of the concentration of the biotin labeled sorting reagent. Cells were then washed to remove any unbound biotin labeled sorting reagent and subsequently contacted with PE labeled streptavidin. Cells expressing antibodies specifically to an HLA-A2/DNTT (250-258) peptide interface remaining bound to the biotin labeled sorting reagent were identified through PE labeled streptavidin.

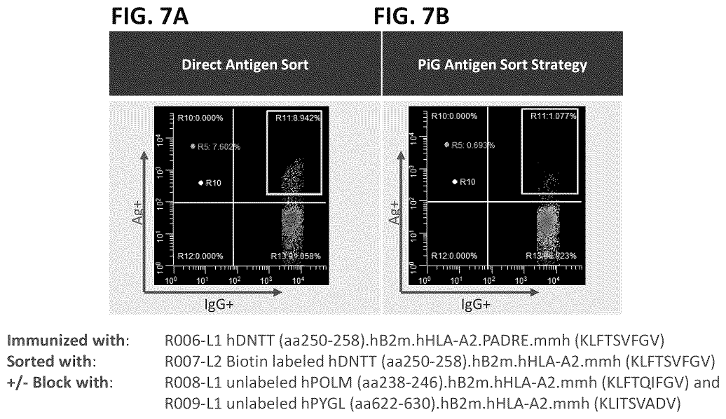

Cells identified by the two methods were then further analyzed using fluorescence activated cell sorting (FACS). As shown in FIG. 7A, 363 B cells out of one million splenocytes expressing antibodies bind to the biotin labeled sort reagent were detected (yellow rectangle) from the mouse using the direct sort method. In contrast, as shown in FIG. 7B, only 54 B cells out of one million splenocytes expressing antibodies specific to the biotin labeled sort reagent were detected (yellow rectangle) using the PiG sort method.

Single B cells that were detected using the PiG sort method were sorted and plated. Each individual B cells were subsequently cloned into expression vectors containing human IgG constant regions. Recombinant antibodies were produced from CHO cells after transient transfection. Supernatant from each CHO clone was subsequently collected for Luminex measurement following the procedure described in Example 1.

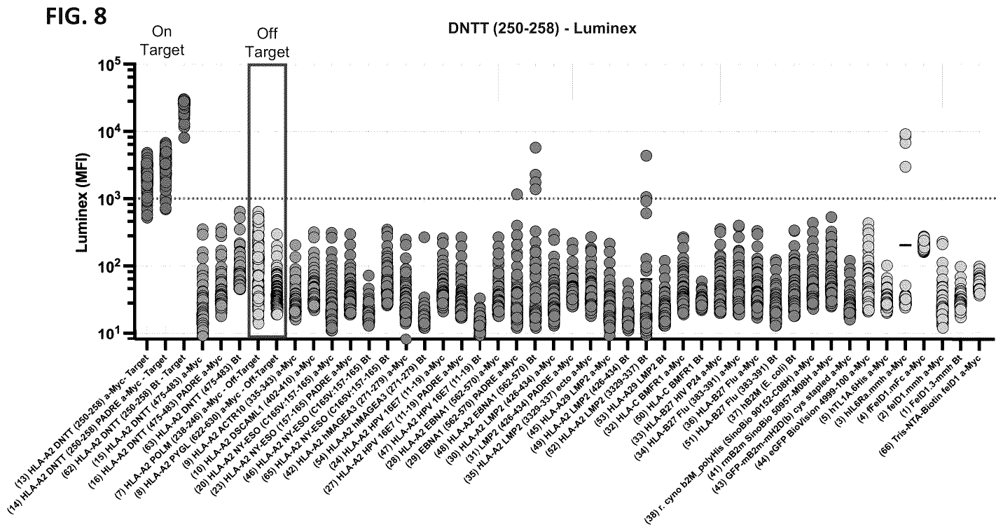

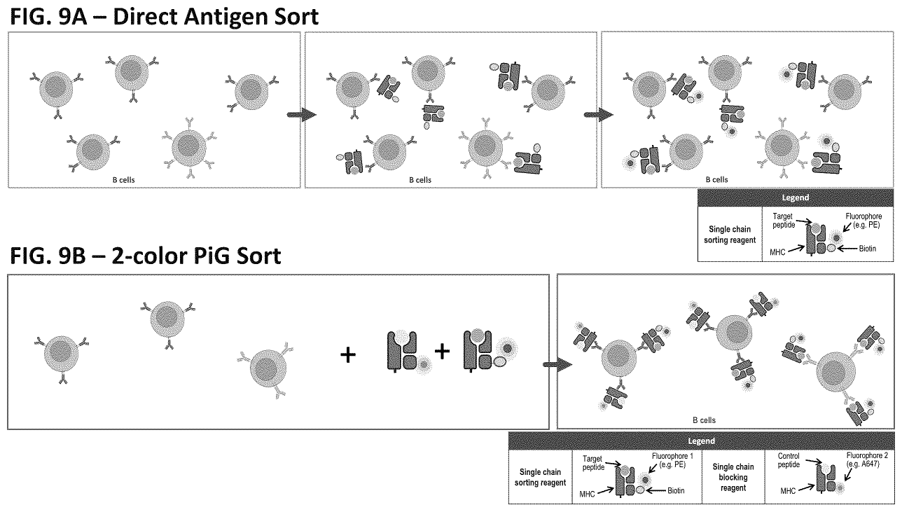

FIG. 8 demonstrates the Luminex binding assay results from the PiG antigen sort method. A series of protein-coupled beads tested in the immunoassay are listed in the X-axis in FIG. 8. Proteins coupled on beads annotated 13, 14, and 62 are the “on-target” DNTT (250-258) peptide-ß2m-HLA-A2 trimer protein with, the rest are the HLA-A2/ß2m trimer proteins with either the “off-target” peptides hPOLM (aa238-246) (KLFTQIFGV, SEQ ID NO: 6) and hPYGL (aa622-630) (KLITSVADV, SEQ ID NO: 7) coupled on beads as annotated 7 and 8 respectively, or the “irrelevant” peptides coupled on beads as annotated 15, 16, 63, 9, 10, 20, 23, 65, 42, 54, 24, 27, 47, 28, 29, 48, 30, 31, 35, 49, 52; or other MHC trimer proteins as annotated 45, 53, 32, 50, 33, 34, 36, 51; or different species beta2-microglobulin (ß2m) proteins as annotated 37, 38, 41; or irreverent control proteins as annotated 43, 44, 5, 3, 4, 2, 1, and 66. Mean fluorescence intensities (MFI) measured below 1,000 are deemed non-specific bindings as denoted below the dotted line in the graph. Among the total 156 clones tested, the majority of the antibodies showed specific bindings (i.e. above 1,000 MFI) toward the two “on-target” HLA-A2/ß2m trimer proteins as colored in blue in the “on-target” rectangle (FIG. 8), while showing no bindings toward the two “off-target” peptide HLA-A2/ß2m trimer proteins (hPOLM (aa238-246) and hPYGL (aa622-630)) as colored in orange, and showing comparably low or no bindings toward the rest of the proteins (colored in grey) including irreverent peptide HLA-A2/ß2m trimer proteins DNTT (475-483), ACTR10 (335-343), DSCAML1 (402-410), NY-ESO (157-165), MAGEA3 (271-279), HPV 16E7 (11-19), EBNA1 (562-570), LMP2 (426-434), and LMP2 (329-337), as well as to other MHC trimer proteins HLA-A29, HLA-B27, HLA-C, and mH2D(b), to human ß2m, monkey ß2m, mouse ß2m proteins, and to several control proteins EGFP, TL1A.6His, IL6Ra, and FelD1. This indicating that the majority of the antibodies specifically bind the epitopes resides in the unique “on-target” DNTT (250-258)-ß2m-HLA-A2 trimer proteins with interfaces, and only a handful of antibodies bind the common epitopes shared between the “on-target” HLA-A2/ß2m trimers and the irrelevant HLA-A2/ß2m trimers. The results showed specific DNTT (250-258) Peptide-in-binding-Groove HLA-A2 complex interface (PiG) selectivity of antibodies isolated from the PiG sort method.

### Example 4: PiG Sort Method for Enriching for Cells Expressing Antibodies Specific to HLA-DQ/Gliadin

This Example describes experiments performed to demonstrate the ability of PiG sort method in enriching for cells expressing antibodies specifically against micro-epitopes of Gliadin (QLQPFPQPELPY, SEQ ID NO: 8) Peptide-in-binding-Groove HLA-DQ MHC class II complex interface.

Mice were immunized with DNA encoding Gliadin-HLA-DQB.PADRE.LCMV and HLA-DQA. The PADRE epitope is described by M. F. del Guercio et al., Potent immunogenic short linear peptide constructs composed of B cell epitopes and Pan DR T helper epitopes (PADRE) for antibody responses in vivo. Vaccine 15, 441-448 (1997). Spleens from the immunized mice were harvested, and splenocytes were obtained and red blood cells were removed by lysis. The cells were then stained with fluorochrome conjugated antibodies specific to B cell surface markers (IgG in this case) to identify B cells.

Antigen expressing B cells were treated using two different methods for comparison: the direct antigen sort method and the PiG sort method.

In the direct antigen sort method used for this Example, antibody-anchored cells were first contacted with biotin labeled Gliadin HLA-DQB.Jun.PADRE.LCMV.HLA-DQA.Fos trimer protein as a sorting reagent. Cells were washed to remove any unbound biotin labeled sorting reagent and then contacted with PE labeled streptavidin. Cells expressing antibodies remaining bound to the biotin labeled sorting reagent were identified through PE labeled streptavidin. This direct antigen sort method would identify cells expressing antibodies to various possible immunogenic epitopes of the sorting reagent Gliadin HLA-DQB.Jun.PADRE.LCMV.HLA-DQA.Fos trimer protein.

In the PiG antigen sort method used for this Example, cells were first contacted with a blocking reagent containing an unlabeled trimer protein HSV (EEVDMTPADALD, SEQ ID NO: 9, as a control peptide)-HLA-DQB.Jun.PADRE.LCMV.HLA-DQA.Fos. After ten to fifteen minutes of pre-incubating with the unlabeled trimer blocking reagent, cells were further exposed to a biotin labeled sort reagent Gliadin HLA-DQB.Jun.PADRE.LCMV.HLA-DQA.Fos in the presence of the unlabeled trimer blocking reagent. The concentration of the blocking reagent used in the preincubation step was forty-fold of the concentration of the biotin labeled sorting reagent. Cells were then washed to remove any unbound biotin labeled sorting reagent and subsequently contacted with PE labeled streptavidin. Cells expressing antibodies specifically to an HLA-DQ/Gliadin peptide interface remaining bound to the biotin labeled sorting reagent were identified through PE labeled streptavidin.

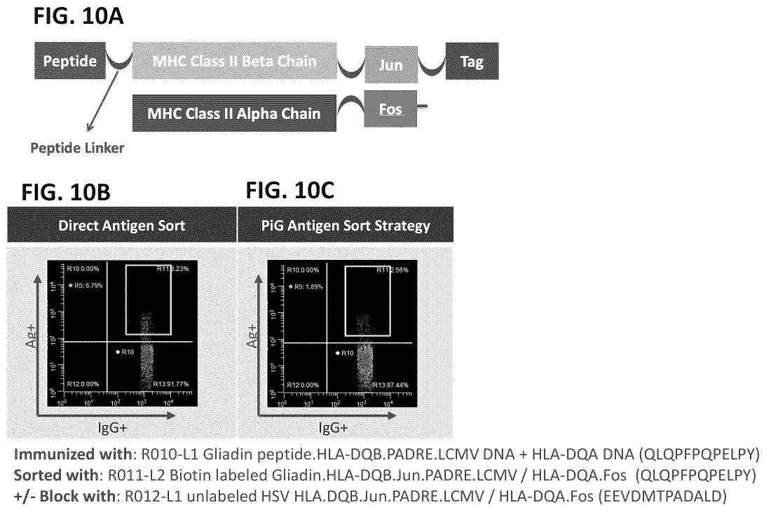

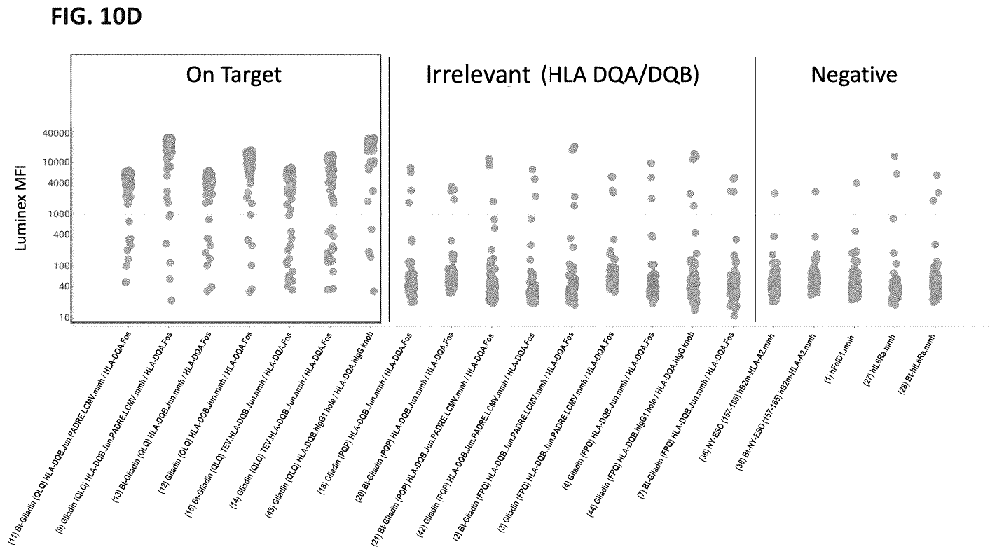

Cells identified by the two methods were then further analyzed using fluorescence activated cell sorting (FACS). As shown in FIG. 10B, 165 B cells out of one million splenocytes expressing antibodies bind to the biotin labeled sort reagent were detected (yellow rectangle) from the mouse using the direct sort method. In contrast, as shown in FIG. 10C, only 92 B cells out of one million splenocytes expressing antibodies specific to the biotin labeled sort reagent were detected (yellow rectangle) using the PiG sort method.

Single B cells that were detected using the PiG sort method were sorted and plated. Each individual B cells were subsequently cloned into expression vectors containing human IgG constant regions. Recombinant antibodies were produced from CHO cells after transient transfection. Supernatant from each CHO clone was subsequently collected for Luminex measurement following the procedure described in Example 1.

FIG. 10D demonstrates the Luminex binding assay results from the PiG antigen sort method. A series of protein-coupled beads tested in the immunoassay are listed in the X-axis in FIG. 10D. Proteins coupled on beads annotated 11, 9, 13, 12, 15, 13, and 43 are the “on-target” HLA-DQA/HLA-DQB trimer proteins with Gliadin peptide, the rest are the HLA-DQA/HLA-DQB trimer proteins with “irrelevant” control peptides coupled on beads as annotated 18, 20, 21, 42, 2, 3, 4, 44 and 7; or other MHC trimer proteins as annotated 36 and 38; or irrelevant control proteins as annotated 1, 27 and 28. Mean fluorescence intensities (MFI) measured below 1,000 are deemed non-specific bindings as denoted below the dotted line in the graph. Among the total 88 clones tested, the majority of the antibodies showed specific bindings (i.e. above 1,000 MFI) toward the seven “on-target” HLA-DQA/HLA-DQB trimer proteins in the red rectangle, as shown in FIG. 10D, from left to right, with 79 clones, 83 clones, 79 clones, 82 clones, 73 clones, 74 clones, and 84, respectively, out of total 88 clones, while comparably low number of antibodies show specific bindings toward the irrelevant peptide HLA-DQA/HLA-DQB trimer proteins including other Gliadin peptides PQPELPYPQPQL (SEQ ID NO: 10) and FPQPEQPFPWQP (SEQ ID NO: 11), as well as to other MHC trimer proteins HLA-A2/ß2m, as well as to several control proteins hFelD1.mmh, IL6Ra.mmh and Bt-IL6Ra.mmh. These results indicate that the majority of the antibodies bind the epitopes residing in the unique “on-target” HLA-DQA/HLA-DQB/Gliadin (QLQPFPQPELPY, SEQ ID NO: 8) interfaces, and only a limited number of antibodies bind the common epitopes shared between the “on-target” HLA-DQA/HLA-DQB trimers and the irrelevant HLA-DQA/HLA-DQB trimers. The results showed specific Gliadin (QLQPFPQPELPY, SEQ ID NO: 8) Peptide-in-binding-Groove HLA-DQ complex interface (PiG) selectivity of antibodies isolated using the PiG sort method.

### Example 5. Preparation of Non-Covalently Associated Peptide-MHC Trimer

This Example describes an exemplary process for preparation of a non-covalently associated peptide-MHC class I trimer.

Expression—An HLA heavy chain with C-terminus BirA tag is expressed in E. coli. B2M (light chain) is also expressed in E. coli. When HLA and B2M were recombinantly expressed in E. coli they form insoluble inclusion bodies.

Isolation of Inclusion Bodies—Inclusion bodies are washed 3 times with 1% Triton X-100 in Tris buffer to remove residual soluble proteins and lipids, followed by 3 washes with buffer alone to remove remaining Triton X-100.

Solubilization of Protein in 8 M urea—Protein is solubilized from inclusion bodies in 8 M urea. This denatures the protein.

Protein Refolding—Urea solubilized HLA heavy chain, urea solubilized B2M light chain and peptide of interest (solubilized in DMSO) are combined together with reagents to help protein folding, disulfide bond formation and suppress protein aggregation in “Rapid Dilution Refold”. Dilution is important to drop urea concentration to below 0.1 M urea in order to allow for protein folding. Refold reaction is incubated for 48 hours and 1 week (increasing time can help yield of refolded complex). Conditions can be tested at a small scale before scaling up.

Refold is concentrated before purifying over Size Exclusion Chromatography—SDS-PAGE confirms presence of heavy and light chain in purified complex. Peptide In Groove Complex will not form without peptide. Presence of peptide is confirmed by mass spec analysis.

Biotinylation of Complex using BirA ligase—Biotinylated complex is purified over Size Exclusion Chromatography to remove excess biotin.

Neutravidin blot confirms biotinylation of complex.

1. A method for isolating antibody-producing cells that express antibody molecules specific for a peptide-MHC interface, the method comprising:
(a) contacting a population of antibody-producing cells encompassing cells that express on the cell surface antibody molecules specific for an interface between a target peptide and an MHC molecule with a blocking reagent, said blocking reagent comprises a peptide-MHC trimer,
(b) contacting the population of cells in the presence of the blocking reagent with a sorting reagent which comprises a labeled peptide-MHC trimer,
(c) wash the population of cells to remove unbound sorting reagent; and
(d) collecting cells that remain bound to the sorting reagent to obtain cells expressing antibody molecules specific for an interface between the target peptide and the MHC molecule,
wherein the peptide-MHC trimer in the blocking reagent comprises a control peptide and two MHC polypeptide chains comprising (i) the α chain or a portion thereof of the MHC molecule and β2 microglobulin or a portion thereof when the MHC molecule is an MHC class I molecule, or (ii) the α chain or a portion thereof and the β chain or a portion thereof of the MHC molecule when the MHC molecule is an MHC class II molecule, wherein the control peptide differs from the target peptide by at least one amino acid, and is presented in a peptide-binding groove formed by the two MHC polypeptide chains;
wherein the peptide-MHC trimer in the sorting reagent comprises the target peptide and two MHC polypeptide chains comprising (i) the α chain or a portion thereof of the MHC molecule and β2 microglobulin or a portion thereof when the MHC molecule is an MHC class I molecule, or (ii) the α chain or a portion thereof and the β chain or a portion thereof of the MHC molecule when the MHC molecule is an MHC class II molecule, wherein the target peptide is presented in a peptide-binding groove formed by the two MHC polypeptide chains; and
wherein the peptide-MHC trimer in the blocking reagent is unlabeled or labeled differently from the labeled peptide-MHC trimer in the sorting reagent.

2. The method of claim 1, wherein the MHC molecule is an MHC class I molecule.

3. The method of claim 1, wherein the MHC molecule is an MHC class II molecule.

4. The method of claim 2, wherein the MHC class I molecule is a human MHC class I molecule.

5. (canceled)

6. The method of claim 3, wherein the MHC class II molecule is a human MHC class II molecule.

7. (canceled)

8. The method of claim 2, wherein in the blocking reagent, the α chain or the portion thereof comprises the extracellular sequence of an MHC class I α chain, and the β2 microglobulin or the portion thereof comprises the mature form of the β2 microglobulin.

9. The method of claim 2, wherein in the sorting reagent, the α chain or the portion thereof comprises the extracellular sequence of the α chain, and the β2 microglobulin or the portion thereof comprises the mature form of the β2 microglobulin.

10. The method of claim 3, wherein in the blocking reagent, the α chain or the portion thereof comprises the extracellular sequence of the α chain, and the β chain or the portion thereof comprises the extracellular sequence of the β chain.

11. The method of claim 3, wherein in the sorting reagent, the α chain or the portion thereof comprises the extracellular sequence of the α chain, and the β chain or the portion thereof comprises the extracellular sequence of the β chain.

12. The method of claim 1, wherein the two polypeptide chains of the MHC molecule in the peptide-MHC trimer in the blocking reagent are identical in sequence to the two polypeptide chains of the MHC molecule in the peptide-MHC trimer in the sorting reagent.

13. The method of claim 1, wherein the control peptide in the blocking reagent is covalently linked to one of the two MHC polypeptide chains, and/or wherein the target peptide in the sorting reagent is covalently linked to one of the two MHC polypeptide chains.

14. The method of claim 2, wherein the control peptide in the blocking reagent is covalently attached to the α chain or the portion thereof or the β2 microglobulin or the portion thereof via a peptide linker.

15. The method of claim 2, wherein the control peptide, the α chain or the portion thereof, and the β2 microglobulin or the portion thereof, are linked in a single chain.

16. The method of claim 2, wherein the target peptide in the sorting reagent is covalently attached to the α chain or the portion thereof or the β2 microglobulin or the portion thereof via a peptide linker.

17. The method of claim 2, wherein the target peptide, the α chain or the portion thereof, and the β2 microglobulin or the portion thereof, are linked in a single chain.

18. The method of claim 3, wherein the control peptide in the blocking reagent is covalently attached to the α chain or the portion thereof or the β chain or the portion thereof via a peptide linker.

19. The method of claim 3, wherein the α chain or the portion thereof and the β chain or the portion thereof are linked by a Jun-Fos zipper comprising a Jun leucine zipper dimerization motif and a Fos leucine zipper dimerization motif.

20. The method of claim 3, wherein the target peptide in the sorting reagent is covalently attached to the α chain or the portion thereof or the β chain or the portion thereof via a peptide linker.

21. The method of claim 3, wherein the α chain or the portion thereof, and the β chain or the portion thereof, are linked by a Jun-Fos zipper comprising a Jun leucine zipper dimerization motif and a Fos leucine zipper dimerization motif.

22. The method of claim 1, wherein the target peptide comprises a tumor associated antigen, a bacterial antigen, or a viral antigen.

23.-26. (canceled)

27. The method of claim 1, wherein the control peptide differs from the target peptide by at least 2 amino acids.

28.-29. (canceled)

30. The method of claim 1, wherein the control peptide is an off-target peptide.

31. The method of claim 1, wherein the blocking reagent comprises two or more peptide-MHC trimers, wherein the control peptides in the two or more peptide-MHC trimers differ from one another.

32. The method of claim 1, wherein the peptide-MHC trimer in the blocking reagent is unlabeled.

33. The method of claim 32, wherein the unlabeled peptide-MHC trimer in the blocking reagent in step (a) has a molar concentration at least 10 fold relative to the molar concentration of the labeled peptide-MHC trimer in the sorting reagent.

34.-36. (canceled)

37. The method of claim 1, wherein the peptide-MHC trimer in the sorting reagent is labeled with a first fluorescent compound, and wherein the peptide-MHC trimer in the blocking reagent is labeled with a second fluorescent compound that differentiates from the first fluorescent compound.

38. The method of claim 37, wherein the peptide-MHC trimer in the blocking reagent is at about the same or higher molar concentration as the peptide-MHC trimer in the sorting reagent.

39. The method of claim 1, wherein the blocking reagent and the sorting reagent are brought into contact with the population of cells at the same time.

40. The method of claim 1, wherein the peptide-MHC trimer in the sorting reagent is labeled indirectly with a fluorescent compound.

41. The method of claim 40, wherein the peptide-MHC trimer in the sorting reagent is conjugated with biotin which binds streptavidin labeled with the fluorescent compound.

42. The method of claim 40, wherein fluorescence-activated cell sorting is used to collect cells that remain bound to the labeled sorting reagent.

43. The method of claim 1, wherein the peptide-MHC trimer in the blocking reagent and/or the sorting reagent is provided in a multimeric form.

44. The of claim 1, wherein the peptide-MHC trimer in the blocking reagent and/or the sorting reagent is provided in a monomeric form.

45. The method of claim 43, wherein the multimeric form is formed by a multivalent molecule to which the peptide-MHC trimer is bound or linked, and wherein the multivalent molecule is selected from a streptavidin multimer such as tetramer, a dimer of an immunoglobulin Fc fragment, or a trimer of a trimerization molecule such as foldon.

46. (canceled)

47. The method of claim 45, wherein the peptide-MHC trimer is conjugated with biotin which binds streptavidin to tetramerize the peptide-MHC trimer, and wherein optionally the streptavidin is labeled with a fluorescent compound.

48. The method of claim 1, wherein the collected cells are sorted to single cells.

49. The method of claim 48, further comprising: isolating nucleic acids that encode the antibody molecules from the single cells.

50. The method of claim 49, further comprising transfecting a host cell with a nucleic acid encoding an antibody heavy chain or variable domain thereof, and a nucleic acid encoding an antibody light chain or variable domain thereof; and growing the transfected host cell under conditions to support expression of antibody by the host cell.

51. The method of claim 50, wherein the host cell is Chinese hamster ovary (CHO) cell.

52. The method of claim 1, wherein the antibody producing cells are primary antibody producing cells, yeast cells, or immortalized mammalian cells which produce antibody molecules on the cell surface.

53. The method of claim 52, wherein the primary antibody-producing cells are obtained from spleen, lymph node, peripheral blood and/or bone marrow of a mammal.

54. The method according to claim 53, wherein the primary antibody-producing cells comprise B cells.

55. The method of claim 53, where the primary antibody-producing cells are obtained from a mouse immunized with an immunogen comprising a peptide-MHC trimer which comprises the target peptide and two polypeptide chains of the MHC molecule, wherein the two polypeptide chains comprises (i) an MHC class I α chain or a portion thereof and β2 microglobulin when the MHC molecule is a MHC class I molecule, or (ii) an MHC class II α chain or a portion thereof and an MHC class II β chain or a portion thereof when the MHC molecule is a MHC class II molecule, wherein the target peptide is presented in a peptide-binding groove formed by the two polypeptide chains of the MHC molecule.

56. The method of claim 55, wherein the two polypeptide chains of the peptide-MHC trimer in the immunogen are identical in sequence to the two polypeptide chains of the peptide-MHC trimer in the sorting reagent.

57. The method according to claim 52, wherein the immortalized mammalian cells which produce antibody molecules are selected from Chinese hamster ovary (CHO) cells and hybridoma cells.
